SEQUENCE LISTING (14 sequences)

SEQ ID NO: 1 - protein, 4 aa, Artificial Sequence
  synthetic sequence
GFLG

SEQ ID NO: 2 - protein, 13 aa, Artificial sequence
  synthetic sequence
GRKKRRQRRRPPQ

SEQ ID NO: 3 - protein, 7 aa, Artificial sequence
  synthetic sequence
GNNQQNY

SEQ ID NO: 4 - protein, 7 aa, Artificial sequence
  synthetic sequence
GVQIVYK

SEQ ID NO: 5 - protein, 4 aa, Artificial sequence
  synthetic sequence
VVVV

SEQ ID NO: 6 - protein, 6 aa, Artificial sequence
  synthetic sequence
VEVEVE

SEQ ID NO: 7 - protein, 6 aa, Artificial sequence
  synthetic sequence
NNQQNY

SEQ ID NO: 8 - protein, 14 aa, Artificial sequence
  synthetic sequence
LLKKLLKLLKKLLK

SEQ ID NO: 9 - protein, 10 aa, Artificial sequence
  synthetic sequence
CGNNQQNYKK

SEQ ID NO: 10 - protein, 9 aa, Artificial sequence
  synthetic sequence
CGVQIVYKK

SEQ ID NO: 11 - protein, 9 aa, Artificial sequence
  synthetic sequence
GNNQQNYKK

SEQ ID NO: 12 - protein, 6 aa, Artificial sequence
  synthetic sequence
VQIVYK

SEQ ID NO: 13 - protein, 11 aa, Artificial sequence
  synthetic sequence
KGNNQQNYKKK

SEQ ID NO: 14 - protein, 11 aa, Artificial sequence
  synthetic sequence
KGNNQQNYEEE

(19) United States
(12) Patent Application Publication
(10) Pub. No.: US 2014/0113322 A1
(43) Pub. Date: Apr. 24, 2014

(54) SUPRAMOLECULAR NANOBEACON IMAGING AGENTS AS PROTEASE SENSORS

(71) Applicant: THE JOHNS HOPKINS UNIVERSITY, BALTIMORE, MD (US)

(72) Inventors: Honggang Cui, Lutherville, MD (US); Andrew G. Cheetham, Arlington, VA (US); Lye Lin Lock, Baltimore, MD (US)

(73) Assignee: THE JOHNS HOPKINS UNIVERSITY, BALTIMORE, MD (US)

(21) Appl. No.: 14/059,974

(22) Filed: Oct. 22, 2013

Related U.S. Application Data
(60) Provisional application No. 61/716,809, filed on Oct. 22, 2012.

(51) Int. Cl. C12Q 1/37

(52) U.S. Cl. CPC C12Q 1/37
USPC 435/23; 530/324

(57) ABSTRACT

Disclosed herein are novel nanobeacon imaging agents having the following formula:

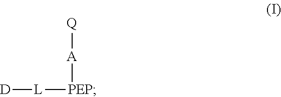

wherein: D is 1 to 4 fluorophores; L is 1 to 4 enzymatically cleavable peptide linkers; PEP is a hydrophilic cell penetrating peptide sequence; A is a side chain moiety of an amino acid of PEP; and Q is a fluorescence quencher molecule. The present invention provides a generic design of a new type of supramolecular nanobeacon imaging agents with a well-defined size and surface chemistry for protease detection. In contrast to soluble molecular beacons, the imaging agent molecules are specifically designed to self-assemble into core-shell micellar structures, with the enzyme-sensitive design feature being deeply embedded within the micellar core and thus inaccessible to the enzyme. Only in the monomeric form can these nanobeacon imaging agent molecules be cleaved by the target enzyme to generate fluorescence signals. In some embodiments, the nanobeacons can be tuned to different shapes depending on the environmental conditions. In other embodiments, the nanobeacons can be linked to a targeting moiety. Methods of use of the imaging agent molecules for in vitro and in vivo research, diagnosis, and treatment, as well as methods of making these imaging agents are also provided.

### REFERENCE TO RELATED APPLICATIONS

This application claims the benefit of U.S. Provisional Patent Application No. 61/716,809, filed on Oct. 22, 2012, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Oct. 22, 2013, is named P12147-02_ST25.txt and is 2,872 bytes in size.

### BACKGROUND OF THE INVENTION

Real time detection of the location and expression level of enzymes within living cells offers important information on many important cellular and subcellular events and thus provides unique opportunities for the development of new strategies for tumor diagnosis and cancer therapeutics. The overexpression and relative abundance of certain proteases in cancers, such as cathepsins and matrix metalloproteases (MMPs), provide attractive targets for tumor screening. In the design of polymer-drug conjugates with peptide linkers, enzymatic cleavage is an important step towards the release of bioactive anticancer drugs, with the release rate being a function of active enzyme concentration. Recently, there is also a rapidly growing interest in the development of enzymatically responsive materials. Therefore, it is important and necessary to precisely detect the activities or expression levels of enzymes of interest.

The advent and development of activatable molecular probes, which are imaging agents or molecular beacons that contain a fluorophore and quencher pair, have enabled possibilities for the highly sensitive detection of DNA/RNA through the conversion of specific binding events into detectable fluorescence signals. Very recently, molecular beacons with proteolytically degradable peptide linkers have been devised for protease detection and other applications. However, since the linkers that are designed to activate molecular beacons are typically exposed to the physiological environment, their poor stability and facile degradation by non-specific enzymes often give rise to an undesired false signal and thus pose a major limitation for accurate detection of enzymatic activities.

Therefore, there still exists a need for novel molecular probe based imaging agents that are capable of prolonged circulation time and resist degradation prior to locating to the target sites of interest, and are capable of quantifying the activity of the target enzymes intracellularly.

### SUMMARY OF THE INVENTION

The present invention describes the generic design and fabrication of a new type of peptide-based supramolecular nanobeacon imaging agents for the detection of protease activity in vitro and in vivo. The nanobeacon imaging agents are activatable through the incorporation of one or more enzymatically cleavable linker groups that connect one or more fluorophores to a quencher-peptide conjugate. The key design principle is that these nanobeacon imaging agents can spontaneously assemble into well-defined supramolecular nanostructures that embed the fluorophore(s) and quencher pair and enzyme-sensitive linker(s) within the core of the assembly, thus shielding it from the physiological environment. Upon localization at the target site or internalization into cells of interest, the nanostructures will dissociate to release monomers that can be cleaved by target proteases, thus generating a measurable fluorescence signal that can be used to identify the location of and/or quantify the activity of the protease.

In accordance with one or more embodiments, the present inventions disclosed herein would significantly improve the detection of proteases upon the previous platforms in five aspects: 1) prolonged circulation time and controlled pharmacokinetics (nanostructures versus individual molecules); 2) more accurate detection of the location and quantity of target enzymes by minimizing non-specific enzyme degradation, as the activatable linkers of imaging agents are deeply embedded inside the cores and thus inaccessible to any enzyme unless they first dissociate into the monomeric form; 3) improved sensitivity due to the fact that the imaging agents of the present invention can serve as a molecular probe reservoir to supply substrate molecules for enzyme cleavage, leading to a very high local concentration of molecular probes in the targeted area. Single enzyme imaging is possible since each spherical imaging agent probe contains approximately 50-70 molecules (cylindrical nanobeacons contain thousands of molecules or more); 4) minimization of toxicity due to the fact that there is no additional nanocarrier needed to deliver the molecular probes since imaging agents are simply formed by self-assembly; and 5) with multiple fluorophores having different wavelengths and having different linkers, more than one protease can be detected.

In accordance with an embodiment, the present invention provides an imaging agent having the following formula (I):

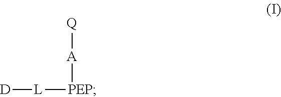

wherein: D is 1 to 4 fluorophores which can be the same or different; L is 1 to 4 enzymatically cleavable peptide linkers which can be the same or different; PEP is a peptide sequence with overall hydrophilicity that both promotes the self-assembly of the designed molecules into nanostructures and facilitates better cell targeting and internalization; A is a side chain moiety of an amino acid of PEP; and Q is a fluorescence quencher molecule.

In accordance with another embodiment, the present invention provides an imaging agent having the following formula (II):

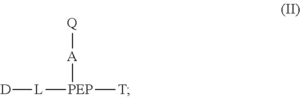

wherein: D is 1 to 4 fluorophores which can be the same or different; L is 1 to 4 enzymatically cleavable peptide linkers which can be the same or different; PEP is a peptide sequence with overall hydrophilicity that both promotes the self-assembly of the designed molecules into nanostructures and facilitates better cell targeting and internalization; A is a side chain moiety of an amino acid of PEP; Q is a fluorescence quencher molecule; and T is a targeting ligand.

In accordance with a further embodiment, the present invention provides an imaging agent having the following formula:

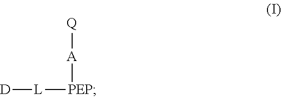

wherein: D is 5-carboxyfluorescein (5-FAM); L is an enzymatically cleavable peptide linker having the sequence GFLG (SEQ ID NO: 1); PEP is a hydrophilic cell penetrating peptide sequence comprising amino acids 48-60 of the HIV Tat protein; A is the side chain moiety of a lysine of PEP; and Q is Black Hole Quencher 1 (BHQ-1).

In accordance with yet another embodiment, the present invention provides the imaging agents identified above wherein the PEP moiety is capable of having different nanostructures depending on temperature and/or pH and/or aging.

In accordance with a further embodiment, the present invention provides a method of identifying a cell or a population of cells in vivo expressing one or more proteases of interest comprising: a) contacting the cell or a population of cells expressing at least one protease of interest with the imaging agent described above, which is selectively cleavable by a protease of interest; b) allowing the imaging agent to be selectively cleaved by the at least one protease of interest in the cell or population of cells; and c) detecting the presence of the fluorescent imaging agent after being cleaved by the at least one protease of interest in the cell or population of cells.

In accordance with a still further embodiment, the present invention provides a method of diagnosing a disease in a patient comprising: a) administering to a patient suspected of having said disease, an imaging agent which is selectively cleavable by at least one protease of interest, the cleavage of which indicates the presence of the disease, wherein said imaging agent is an imaging agent described above; b) allowing the imaging agent to be cleaved by the at least one protease of interest; and c) detecting the presence of the imaging agent binding the protease of interest in the patient.

In accordance with yet another embodiment, the present invention provides a method of identifying a cell or a population of cells in vivo expressing a cathepsin B comprising: a) contacting the cell or a population of cells expressing cathepsin B with imaging agent having the following formula:

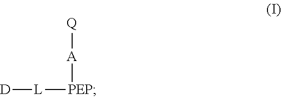

wherein: D is 5-carboxyfluorescein (5-FAM); L is an enzymatically cleavable peptide linker having the sequence GFLG (SEQ ID NO: 1); PEP is a hydrophilic cell penetrating peptide sequence comprising amino acids 48-60 of the HIV Tat protein; A is the side chain moiety of a lysine of PEP; and Q is Black Hole Quencher 1 (BHQ-1); b) allowing the imaging agent to be selectively cleaved by cathepsin B in the cell or population of cells; and c) detecting the presence of the fluorescent imaging agent after being cleaved by cathepsin B in the cell or population of cells.

### BRIEF DESCRIPTION OF THE DRAWINGS

FIG. 1 is a schematic illustration of the expected cleavage and detection mechanism (A) and molecular structure of one embodiment of the designed nanobeacon imaging agent (designated as “TFB”) (B). In the self-assembled state, the enzyme-sensitive linker is deeply buried in the micellar core. When in the monomeric form, the imaging agent molecules become accessible for enzymatic cleavage. The transition from imaging agent micelles to monomeric forms can be achieved either by dilution or by pH triggering.

FIG. 2 depicts photographs of 200 μM aqueous solutions of TFB (A), a control molecule (designated as “TF”) having only the Tat peptide and 5-FAM fluorophore (B), and a second control molecule (designated as “TB”) having only the Tat peptide and the quencher molecule BHQ-1 (C), and their respective molecular structures. The effective quenching of 5-FAM fluorophore by the BHQ-1 segment is reflected in the dramatic color change from bright green (B) to dark red (A). (D) 5-FAM fluorescence measurements of 1 μM TF and 1 μM TFB aqueous solutions.

FIG. 3 depicts the general synthetic scheme for the imaging agents of the present invention, TFB molecule.

FIG. 4 depicts the general synthetic scheme for the imaging agents of the present invention, TF molecule.

FIG. 5 depicts the general synthetic scheme for the imaging agents of the present invention, TB molecule. Abbreviations for FIGS. 3-5: HBTU: O-benzotriazole-N,N,N′,N′-tetramethyluroniumhexafluorophosphate; DIEA: diisopropylethylamine; Mtt: 4-methyltrityl; TFA: trifluoroacetic acid; TIS: triisopropylsilane; DCM: dichloromethane; DMF: N,N-dimethylformamide.

FIG. 6 depicts MALDI-ToF spectra of (a) TFB, (b) TF and (c) TB molecules.

FIG. 7 depicts reverse-phase analytical HPLC of (a) TFB, (b) TF and (c) TB molecules.

FIG. 8 is a plot of TFB surface tension versus log of concentrations. The intersection of two different slopes of lines indicates TFB critical aggregation concentration around 30 μM.

FIG. 9 is a series of TEM (A) and cryo-TEM (B) images of 200 μM TFB in 1×PBS solutions revealing self-assembled nanoparticles of a uniform size (11.1±1.2 nm). TEM images of nanoparticles formed by self-assembly of 400 μM TF (C) and TB (D) in 1×PBS solutions with sizes of 18.4±3.7 nm, and 13.1±1.0 nm, respectively. TEM samples in (A), (C) and (D) were negatively stained using a 2 wt % uranyl acetate aqueous solution to enhance the image contrast. All scale bars: 50 nm.

FIG. 10 depicts circular dichroism spectra of (a) TFB, (b) TF and (c) TB molecules at 100 μM. The measurements were performed at room temperature. Spectra showed a negative peak at 199 nm and positive peak at 219 nm, correlating well with literature values of random-coil secondary structure.

FIG. 11 depicts fluorescence monitoring of the degradation process of imaging agents of the present invention by CatB. (A) Time-course fluorescence measurements of a 3 μM TFB in the presence of 1 μM CatB, pH 5 solution; (B) photographs of NB solutions before and after CatB cleavage; (C) fluorescent measurement of 1 μM TFB PBS solutions in the presence of various concentrations of CatB; (D) plot of initial rate of 5-FAM cleavage versus CatB concentration (square, 1 μM TFB; circle, 50 μM TFB). The red and blue lines show a linear fit for the obtained data.

FIG. 12 is a series of photomicrographs showing time-dependent fluorescence of the imaging agent molecules of the present invention inside MCF-7 human breast cancer cells. Fluorescence images of cells after 0 h (A), 0.5 h (B) and 1.5 h (C) exposure to TFB NB show increased 5-FAM fluorescence with time. The cell nuclei were stained with the blue dye Hoechst 33342. (D) Flow cytometry confirms the increased fluorescence intensity with time inside live MCF-7 cells.

FIG. 13 shows a series of confocal fluorescent images of MCF-7 cells after 2.5 h incubation with NB molecules show colocalization of the fluorescence signal of 5-FAM with that of the Lysotracker Red. (A) Image of 5-FAM fluorescence. (B) Image of Lysotracker Red fluorescence, and (C) a merged image of (A) and (B). The cell nuclei were stained with the blue dye Hoechst 33342.

FIG. 14 is a schematic showing the chemical structure of (a) positively charged SFB-K and (b) negatively charged SFB-E nanobeacon. (c) The self-assembly of SFB molecules were conducted in different temperatures to obtain spherical and cylindrical-shaped nanostructures. With the ability to control nanostructure's charge and shape concomitantly, in-vitro cell studies were conducted to investigate the effect of shape and charge in cellular uptake.

FIG. 15 depicts the general synthetic scheme for the imaging agents of the present invention, SFB-K molecule.

FIG. 16 depicts the general synthetic scheme for the imaging agents of the present invention, SFB-E molecule. Abbreviations for FIGS. 15-16: HBTU: O-benzotriazole-N,N,N′,N′-tetramethyluroniumhexafluorophosphate; DIEA: diisopropylethylamine; Mtt: 4-methyltrityl; TFA: trifluoroacetic acid; TIS: triisopropylsilane; DCM: dichloromethane; DMF: N,N-dimethylformamide.

FIG. 17 depicts MALDI-ToF spectra of (a) SFB-K and (b) SFB-E molecules.

FIG. 18 depicts reverse-phase analytical HPLC of (a) SFB-K and (b) SFB-E molecules.

FIG. 19 is a series of regular TEM images of self-assembled spherical and cylindrical nanostructures formed by SFB-K (a,d), SFB-E (b,e) and SFB-KE (e,f) at 200 μM. Spherical nanostructures were kept at 4° C. while cylindrical nanostructures were aged for more than 4 days at room temperature, in dark. All samples were pre-treated with HFIP and reconstituted in 25 mM HEPES buffer, except cylindrical SFB-E was directly dissolved in 1×DPBS from its lyophilized powder form. Cryo-TEM images of cylindrical SFB-K (g), SFB-E (h), and SFB-KE (i) clearly showed elongated fibers with micro-meter in length.

FIG. 20 depicts PC3-Flu cells incubated with 5 μM of SFB nanobeacons for 60 minutes and the cellular uptake rate of nanobeacons were compared by measuring each cell's fluorescence intensity. (a) Spherical SFB-K showed highest fluorescence intensity followed by SFB-K monomers state. Upon inhibition of energy-dependent endocytosis pathway (+i), PC3-Flu cells did not show appreciable uptake of SFB nanobeacons. (b) Zeta potential measurement of SFB-K nanostructures showed positive surface charge while SFB-E and SFB-KE nanostructures showed an overall of negative surface charge.

FIG. 21 depicts the activation of SFB nano-beacons were actuated by the degradation of Cathepsin-B on -GFLG- linker, which release 5-FAM from FRET quenching in its native form. The enzymatic fluorescence kinetics of (5 μM) SFB-K (a), SFB-E (b), and SFB-KE (c) showed increase in fluorescence intensity after incubated with (+) 0.1 Unit of Cathepsin-B while the fluorescence intensity of nano-beacons without (−) Cathepsin-B remained close to the baseline. After 60 minutes of activation, the fluorescence intensity of each samples were plotted in (d) and the cylindrical nano-beacon showed lower fluorescence intensity than its counterparts.

FIG. 22 shows confocal laser scanning microscopy of PC3-Flu cells after 60 minutes of incubation with 5 μM of SFB nanobeacons in different shapes and charges. Cell nuclei were stained with blue dye Hoechst 33342 and released 5-FAM fluoresced in green. Scale bar: 20 μm.

FIG. 23 depicts confocal microscopy images of (a) released 5-FAM (green channel) and (b) Lysotracker Red staining lysosome (red channel) of PC3-Flu cells after incubated with 5 μM of spherical SFB-K for 60 minutes. (c) Overlay of green and red channels showed co-localization of released 5-FAM in lysosome and DIC image of PC3-Flu cells. (e-f) Co-localization of green and red channels was quantified and the overlap coefficient, R was determined to be 0.9, which indicated high correlation of released 5-FAM located in lysosome. Scale bar: 20 μm.

### DETAILED DESCRIPTION OF THE INVENTION

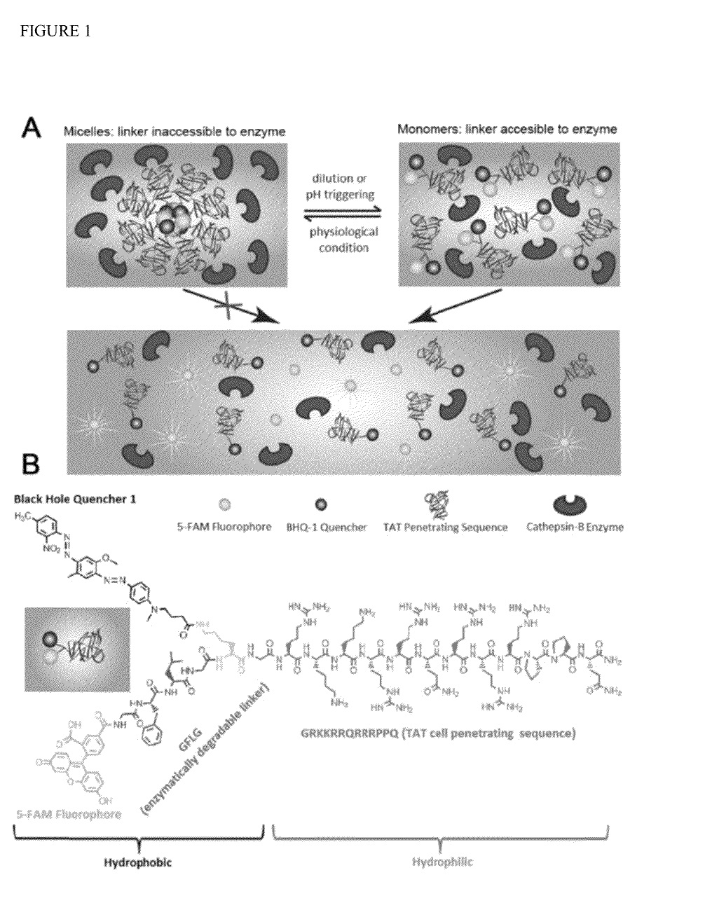

In order to develop molecular probes immune to undesired degradation, the present invention provides a generic design of a new type of supramolecular nanobeacon imaging agent with a well-defined size and surface chemistry for protease detection. In contrast to soluble molecular beacons, the imaging agent molecules are specifically designed to self-assemble into core-shell micelles, with the enzyme-sensitive design feature being deeply embedded within the micellar core and thus inaccessible to the enzyme (FIG. 1A). Only in the monomeric form can these nanobeacon imaging agent molecules be cleaved by the target enzyme to generate fluorescence signals.

The core concept of the imaging agents of the present invention is the construction of an amphiphilic nanobeacon imaging agent molecule having the potential to self-assemble into nano-objects under physiological conditions. This amphiphilicity is achieved by conjugating a hydrophobic quencher and a fluorescent dye onto a hydrophilic peptide. The concept of attaining amphiphilicity by means of conjugating two or more small-molecular chemical moieties with distinct solvent selectivity has been used to successfully construct peptide amphiphiles, peptide nucleic acid amphiphiles, and amphiphilic molecules with π-conjugated segments. FIG. 1B shows the chemical structure of one embodiment of a nanobeacon imaging agent provided herein.

Certain embodiments of the invention can, with appropriate choice of peptide or environmental conditions, undergo morphological transitions from a monomeric state to a variety of nanostructures such as spheres and cylinders. Assembly from monomers into nanostructures can be triggered by 1) an increase in concentration above a critical micellization concentration or 2) a change in pH such that electrostatic repulsions between charge amino acids are minimized, i.e. a higher pH for lysine-based nanobeacons or a lower pH for glutamate-based nanobeacons. The nanostructure morphology can also be tuned through the choice of hydrophilic peptide and by modifying the assembly conditions, e.g. a beta-sheet peptide-containing nanobeacon was found to give spherical structures when assembled at 4° C. from a monomeric state, whereas at room temperature cylindrical structures were formed.

The hydrophobic units in certain embodiments, are composed of a green dye, 5-carboxyfluorescein (5-FAM), and a black hole quencher, BHQ-1, although a number of acceptable fluorophores and quenchers could be used. 5-FAM was chosen on the basis of its exceptionally high quantum yield in the visible light region. The BHQ-1 segment with broad absorption between 400-650 nm (major absorption between 480-580 nm) will, when placed in close proximity to 5-FAM, quench the 5-FAM fluorescence without generating fluorescence of its own, thereby offering a high signal-to-noise ratio. A cell penetrating peptide, for example, HIV-1 derived Tat₄₈₋₆₀, with positively charged arginine and lysine amino acids, was incorporated as the hydrophilic segment to allow effective penetration of the cell membrane. The weakly basic nature of the arginine and lysine residues allows for the design of pH-responsive supramolecular nanobeacon imaging agents. Finally, a key critical component is the cleavable linker that bridges 5-FAM and the lysine N-terminus. In certain embodiments, the peptide tetramer of -Gly-Phe-Leu-Gly- (GFLG) (SEQ ID NO: 1), first identified by Kopecek, Duncan and coworkers (Macromol. Chem. Phys. 1983, 184, 1997-2008; Macromol. Chem. Phys. 1983, 184, 2009-2020) can be effectively cleaved by cathepsin B (CatB), a lysosomal protease involved in cellular protein turnover and degradation. CatB was chosen because it plays important roles in tumor growth and progression and serves as a potential marker for tumor screening. CatB has also attracted considerable interest as the target enzyme in the design of many polymer-drug conjugates.

The present invention provides an imaging agent having the following formula (I)

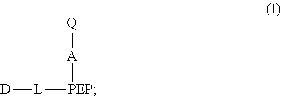

wherein: D is 1 to 4 fluorophores which can be the same or different; L is 1 to 4 enzymatically cleavable peptide linkers which can be the same or different; PEP is a peptide sequence with overall hydrophilicity that both promotes the self-assembly of the designed molecules into nanostructures and facilitates better cell targeting and internalization; A is a side chain moiety of an amino acid of PEP; and Q is a fluorescence quencher molecule.

In accordance with another embodiment, the present invention provides an imaging agent having the following formula (II):

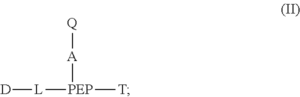

wherein: D is 1 to 4 fluorophores which can be the same or different; L is 1 to 4 enzymatically cleavable peptide linkers which can be the same or different; PEP is a peptide sequence with overall hydrophilicity that both promotes the self-assembly of the designed molecules into nanostructures and facilitates better cell targeting and internalization; A is a side chain moiety of an amino acid of PEP; Q is a fluorescence quencher molecule; and T is a targeting ligand.

As used herein, the term “fluorophore” is understood to mean a fluorochrome, a dye molecule, an organic or inorganic fluorophore, or metal chelate covalently linked to the cleavable peptide linker. A fluorophore can include a far-red or a near-infrared fluorophore. As used herein, the term “fluorophore” means any molecule which can emit a fluorescent signal when excited by the appropriate excitation wavelength. In an embodiment, the fluorophore is a fluorescent dye. There can be up to 4 fluorophores and they can be the same or different, i.e, they can have different excitation or emission wavelengths. The dyes may be emitters in the visible or near-infrared (NIR) spectrum. Known dyes useful in the present invention include carbocyanine, indocarbocyanine, oxacarbocyanine, thiocarbocyanine and merocyanine, polymethine, coumarine, rhodamine, xanthene, fluorescein, borondipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS. In an embodiment, a preferred fluorescent dye is 5-carboxyfluorescine (5-FAM).

Organic dyes which are active in the NIR region are known in biomedical applications. However, there are only a few NIR dyes that are readily available due to the limitations of conventional dyes, such as poor hydrophilicity and photostability, low quantum yield, insufficient stability and low detection sensitivity in biological system, etc. Significant progress has been made on the recent development of NIR dyes (including cyanine dyes, squaraine, phthalocyanines, porphyrin derivatives and BODIPY (borondipyrromethane) analogues) with much improved chemical and photostability, high fluorescence intensity and long fluorescent life. Examples of NIR dyes include cyanine dyes (also known as polymethine cyanine dyes) are small organic molecules with two aromatic nitrogen-containing heterocycles linked by a polymethine bridge and include Cy5, Cy5.5, Cy7 and their derivatives. Squaraines (often called Squarylium dyes) consist of an oxocyclobutenolate core with aromatic or heterocyclic components at both ends of the molecules, an example is KSQ-4-H. Phthalocyanines, are two-dimensional 18π-electron aromatic porphyrin derivatives, consisting of four bridged pyrrole subunits linked together through nitrogen atoms. BODIPY (borondipyrromethane) dyes have a general structure of 4,4′-difluoro-4-bora-3a,4a-diaza-s-indacene) and sharp fluorescence with high quantum yield and excellent thermal and photochemical stability.

In certain embodiments, a fluorescent quencher molecule is used to quench the fluorescent signal from the fluorophore covalently linked to the peptide sequence. For example, an agent can be designed such that the quencher quenches the fluorescence of the fluorophore of the imaging agent when the agent is in an unactivated state, so that the imaging agent exhibits little or no signal until it is activated. It is understood that the quencher can be a non-fluorescent agent, which when suitably located relative to a fluorophore (i.e., at a fluorescence-quenching permissive location) is capable of quenching the emission signal from the fluorophore. As discussed above, it is understood that certain of the foregoing fluorophores can act to quench the fluorescent signal of another spaced apart fluorophore, when the two fluorophores are positioned at fluorescence-quenching interaction permissive locations.

As used herein, the term “quench” is understood to mean the process of partial or complete reduction of the fluorescent signal from a fluorophore. For example, a fluorescent signal can be reduced inter-molecularly or intra-molecularly through the placement of another fluorophore (either the same or a different fluorophore) in fluorescent quenching proximity to the first fluorophore or the placement of a non-fluorogenic quenching chromophore molecule (quencher) in fluorescent quenching proximity to the first fluorophore. The agent is de-quenched (or activated), for example, through the enzymatic cleavage of a peptide sequence.

A number of quenchers are available and known to those skilled in the art including, but not limited to 4-{[4-(dimethylamino)-phenyl]-azo}-benzoic acid (DABCYL), QSY®-7 (9-[2-[(4-carboxy-1-piperidinyl)sulfonyl]phenyl]-3,6-bis(methylphenylamino)-xanthylium chloride) (Molecular Probes, Inc., OR), QSY®-33 (Molecular Probes, Inc., OR), ATTO612Q, ATTO580Q (ATTO-TEC, Germany); Black Hole Quenchers® (Bioresearch Technologies, Novato, Calif.), QXL™680 Acid (AnaSpec, San Jose Calif.), and fluorescence fluorophores such as Cy5 and Cy5.5 (e.g., 2-[5-[3-[6-[(2,5-dioxo-1-pyrrolidinyl)oxy]-6-oxohexyl]-1,3-dihydro-1,1-dimethyl-6,8-disulfo-2H-benz[e]indol-2-ylidene]-1,3-pentadienyl]-3-ethyl-1,1-dimethyl-6,8-disulfo-1H-benz[e]indolium, inner salt) (Schobel, Bioconjugate 10: 1107, 1999), fluorescein isothiocyanates (FITC) and rhodamine pairs. In a preferred embodiment, the quencher is BHQ-1.

As used herein, the term “enzymatically cleavable linker” refers to a peptide fragment that is capable of covalently linking the fluorescent dye molecule to the hydrophilic quencher-containing peptide in the present invention and will be cleaved by a target enzyme, such that the fluorescent dye and quencher molecules will be separated upon cleavage. The linkers are understood to mean a peptide substrate comprising two or more amino acids (as defined herein) that are linked by means of an enzymatically cleavable peptide bond. Also included are moieties that include a pseudopeptide or peptidomimetic. Examples of cleavable peptide substrates can be found in U.S. Pat. No. 7,439,319. In a preferred embodiment, the enzymatically cleavable linker comprises GFLG (SEQ ID NO: 1).

It will be understood by those of ordinary skill in the art that the enzymatically cleavable linker may be introduced either directly as part of the PEP sequence or via common bioconjugation techniques, such as reaction with a cysteine thiol (thiol-ene reaction, disulfide formation, thioether formation) or through Click reactions such as azide-alkyne cycloaddition.

In certain embodiments, the enzymatically cleavable linker is cleavable by at least one enzyme selected from the protease family of enzymes consisting of serine proteases, threonine proteases, cysteine proteases, aspartate proteases, glutamate proteases and metalloproteases. Examples of these include, but are not limited to, cathepsins, matrix metalloproteases (MMPs), caspases or carboxypeptidases.

By extension the present invention may also incorporate a linker that is cleavable by other non-protease enzymes, including but not limited to, glycosidases, lipases, phospholipases, phosphatases, phosphodiesterases, sulfatases, reductases, or bacterial enzymes.

The term “amino acid” as used herein is understood to mean an organic compound containing both a basic amino group and an acidic carboxyl group. Included within this term are natural amino acids (e.g., L-amino acids), modified and unusual amino acids (e.g., D-amino acids), as well as amino acids which are known to occur biologically in free or combined form but usually do not occur in proteins. Natural amino acids include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Other amino acids include, but not limited to, arginosuccinic acid, citrulline, cysteine sulfinic acid, 3,4-dihydroxyphenylalanine, homocysteine, homoserine, ornithine, camitine, selenocysteine, selenomethionine, 3-monoiodotyrosine, 3,5-diiodotryosine, 3,5,5′-triiodothyronine, and 3,3′,5,5′-tetraiodothyronine.

As used herein, the term “peptide sequence with overall hydrophilicity” means a peptide sequence of one or more amino acids which are hydrophilic overall in character and which are capable of forming a nanoparticle or nanofilamentous (such as fiber, ribbon, belt, tube) structure when covalently linked to a hydrophobic quencher and fluorophore in an aqueous solution. In an embodiment, the hydrophilic cell penetrating peptide sequence is the peptide derived from amino acids 48-60 of the HIV-1 Tat protein (GRKKRRQRRRPPQ SEQ ID NO: 2). It will be understood by those of ordinary skill that the sequence is not limited to the particular embodiment, and that other sequences can provide the similar structure necessary for the present invention. The peptide sequences useful in the present invention include, but are not limited to, beta-sheet forming peptides, either from national amyloid protein fragments such as Sup35 (GNNQQNY SEQ ID NO: 3), Tau (GVQIVYK SEQ ID NO: 4), or de novo designed sequences such as VVVV (SEQ ID NO: 5), VEVEVE (SEQ ID NO: 6), collagen peptides, coiled-coil sequences, and random coils.

It will be understood by those of ordinary skill in the art that other peptide fragments which are hydrophilic and which can form a beta-sheet or other secondary structure conformations can also be used in the compositions of the present invention. Examples of hydrophilic peptides include, but are not limited to, NNQQNY (SEQ ID NO: 7) (from the Sup35 yeast prion) and derivatives thereof, GRKKRRQRRRPPQ (SEQ ID NO: 2) (from the HIV Tat protein) and derivatives thereof, LLKKLLKLLKKLLK (SEQ ID NO: 8) (alpha helical peptide) and derivatives thereof, and de novo sequences such as those that possess alternate hydrophobic and hydrophilic residues.

In one or more additional embodiments, the PEP portion of the imaging agents of the present invention is selected from the following peptide sequences: GVQIVYKK (SEQ ID NO: 4); NNQQNY (SEQ ID NO: 7); GRKKRRQRRRPPQ (SEQ ID NO: 2); LLKKLLKLLKKLLK (SEQ ID NO: 8); CGNNQQNYKK (SEQ ID NO: 9); CGVQIVYKK (SEQ ID NO: 10); GNNQQNYKK (SEQ ID NO: 11); (GNNQQNY) (SEQ ID NO: 3) and (VQIVYK) (SEQ ID NO: 12) and derivatives thereof, wherein the derivatives comprise 1 to 10 additional amino acids on either the N-terminal or C-terminal end of PEP.

In accordance with some embodiments, the PEP portion of the imaging agents of the present invention is SUP35K (KGNNQQNYKKK) (SEQ ID NO: 13) or SUP35E (KGNNQQNYEEE) (SEQ ID NO: 14).

As used herein, the term “linking amino acid” means an amino acid that covalently links the hydrophobic quencher to the PEP peptide sequence via a side chain moiety and to the enzymatically cleavable linker. It will be understood by those of skill in the art that any amino acid that can be used in a conjugation reaction can be used as a linking amino acid in the present invention, such as, for example, lysine, cysteine, glutamic acid, aspartic acid, serine or threonine. In a preferred embodiment, the linking amino acid is lysine.

Modified or unusual amino acids which can be used to practice the invention include, but are not limited to, D-amino acids, hydroxylysine, dehydroalanine, pyrrolysine, 2-aminoisobutyric acid, gamma aminobutyric acid, 5-hydroxytryptophan, S-adenosyl methionine, S-adenosyl homocysteine, 4-hydroxyproline, an N-Cbz-protected amino acid, 2,4-diaminobutyric acid, homoarginine, norleucine, N-methylaminobutyric acid, naphthylalanine, phenylglycine, β-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

As used herein, a “pseudopeptide” or “peptidomimetic” is a compound which mimics the structure of an amino acid residue or a peptide, for example, by using linking groups other than via amide linkages (pseudopeptide bonds) and/or by using non-amino acid substituents and/or a modified amino acid residue. A “pseudopeptide residue” means that portion of a pseudopeptide or peptidomimetic that is present in a peptide. The term “pseudopeptide bonds” includes peptide bond isosteres which may be used in place of or as substitutes for the normal amide linkage. These substitute or amide “equivalent” linkages are formed from combinations of atoms not normally found in peptides or proteins which mimic the spatial requirements of the amide bond and which should stabilize the molecule to enzymatic degradation. The following conventional three-letter amino acid abbreviations are used herein: Ala=alanine; Aca=aminocaproic acid, Ahx=6-aminohexanoic acid, Arg=arginine; Asn=asparagines; Asp=aspartic acid; Cha=cyclohexylalanine; Cit=citrulline; Cys=cysteine; Dap=diaminopropionic acid; Gln=glutamine; Glu=glutamic acid; Gly=glycine; His=histidine; Ile=isoleucine; Leu=leucine; Lys=lysine; Met=methionine; NaI=naphthylalanine; Nle=norleucine; Om=ornithine; Phe=phenylalanine; Phg=phenylglycine; Pro=praline; Sar=sarcosine; Ser=serine; Thi=Thienylalanine; Thr threonine; Trp=tryptophan; Tyr=tyrosine; and Val=valine. Use of the prefix D- indicates the D-isomer of that amino acid; for example D-lysine is represented as D-Lys.

The peptides can be synthesized using either solution phase chemistry or solid phase chemistry or a combination of both (Albericio, Curr. Opinion. Cell Biol., 8, 211-221 (2004), M. Bodansky, Peptide Chemistry: A Practical Textbook, Springer-Verlag; N. L. Benoiton, Chemistry of Peptide Synthesis, 2005, CRC Press).

Selective or orthogonal amine protecting groups may be required to prepare the agents of the invention. As used herein, the term “amine protecting group” means any group known in the art of organic synthesis for the protection of amine groups. Such amine protecting groups include those listed in Greene, “Protective Groups in Organic Synthesis” John Wiley & Sons, New York (1981) and “The Peptides: Analysis, Synthesis, Biology, Vol. 3, Academic Press, New York (1981). Any amine protecting group known in the art can be used. Examples of amine protecting groups include, but are not limited to, the following: 1) acyl types such as formyl, trifluoroacetyl, phthalyl, and p-toluenesulfonyl; 2) aromatic carbamate types such as benzyloxycarbonyl (Cbz or Z) and substituted benzyloxycarbonyls, 1-(p-biphenyl)-1-methylethoxycarbonyl, and 9-fluorenylmethyloxycarbonyl (Fmoc); 3) aliphatic carbamate types such as tert-butyloxycarbonyl (Boc), ethoxycarbonyl, diisopropylmethoxycarbonyl, and allyloxycarbonyl; 4) cyclic alkyl carbamate types such as cyclopentyloxycarbonyl and adamantyloxycarbonyl; 5) alkyl types such as triphenylmethyl and benzyl; 6) trialkylsilane such as trimethylsilane; and 7) thiol containing types such as phenylthiocarbonyl and dithiasuccinoyl. Also included in the term “amine protecting group” are acyl groups such as azidobenzoyl, p-benzoylbenzoyl, o-benzylbenzoyl, p-acetylbenzoyl, dansyl, glycyl-p-benzoylbenzoyl, phenylbenzoyl, m-benzoylbenzoyl, benzoylbenzoyl. Other exemplary enzymatically cleavable oligopeptides include a Cys-S—S-Cys moiety.

The present invention provides methods for in vitro and in vivo imaging using the imaging agents disclosed herein. For a review of optical imaging techniques, see, e.g., Alfano et al., Ann. NY Acad. Sci. 820:248-270 (1997); Weissleder, Nature Biotechnology 19, 316-317 (2001); Ntziachristos et al., Eur. Radiol. 13:195-208 (2003); Graves et al., Curr. Mol. Med. 4:419-430 (2004); Citrin et al., Expert Rev. Anticancer Ther. 4:857-864 (2004); Ntziachristos, Ann. Rev. Biomed. Eng. 8:1-33 (2006); Koo et al., Cell Oncol. 28:127-139 (2006); and Rao et al., Curr. Opin. Biotechnol. 18:17-25 (2007).

Optical imaging includes all methods from direct visualization without use of any device and use of devices such as various scopes, catheters and optical imaging equipment, for example computer based hardware for tomographic presentations. The imaging agents are useful with optical imaging modalities and measurement techniques including, but not limited to: endoscopy; fluorescence endoscopy; luminescence imaging; time resolved transmittance imaging; transmittance imaging; nonlinear microscopy; confocal imaging; acousto-optical imaging; photoacoustic imaging; reflectance spectroscopy; spectroscopy; coherence interferometry; interferometry; optical coherence tomography; diffuse optical tomography and fluorescence mediated molecular tomography (continuous wave, time domain frequency domain systems and early photon), and measurement of light scattering, absorption, polarization, luminescence, fluorescence lifetime, quantum yield, and quenching.

An imaging system useful in the practice of the invention typically includes three basic components: (1) an appropriate light source for inducing excitation of the imaging agent, (2) a system for separating or distinguishing emissions from light used for fluorophore excitation, and (3) a detection system. The detection system can be hand-held or incorporated into other useful imaging devices, such as intraoperative microscopes. Exemplary detection systems include an endoscope, catheter, tomographic system, hand-held imaging system, or an intraoperative microscope.

Preferably, the light source provides monochromatic (or substantially monochromatic) light. The light source can be a suitably filtered white light, i.e., bandpass light from a broadband source. For example, light from a 150-watt halogen lamp can be passed through a suitable bandpass filter commercially available from Omega Optical (Brattleboro, Vt.). Depending upon the system, the light source can be a laser. See, e.g., Boas et al., Proc. Natl. Acad. Sci. USA 91:4887-4891, 1994; Ntziachristos et al., Proc. Natl. Acad. Sci. USA 97:2767-2772, 2000; and Alexander, J. Clin. Laser Med. Surg. 9:416-418, 1991. Information on lasers for imaging can be found, for example, at Imaging Diagnostic Systems, Inc., Plantation, Fla. and various other sources. A high pass or bandpass filter can be used to separate optical emissions from excitation light. A suitable high pass or bandpass filter is commercially available from Omega Optical, Burlington, Vt.

In general, the light detection system can be viewed as including a light gathering/image forming component and a light/signal detection/image recording component. Although the light detection system can be a single integrated device that incorporates both components, the light gathering/image forming component and light detection/image recording component.

A variety of light detection/image recording components, e.g., charge coupled device (CCD) systems or photographic film, can be used in such systems. The choice of light detection/image recording depends on factors including the type of light gathering/image forming component being used. It is understood, however, that the selection of suitable components, assembling them into an optical imaging system, and operating the system is within ordinary skill in the art.

With respect to optical in vivo imaging, such a method comprises (a) administering to a subject one or more imaging agents; (b) allowing the agent(s) to distribute within the subject; (c) exposing the subject to light of a wavelength absorbable by at least one fluorophore in the imaging agent; and (d) detecting an optical signal emitted by the fluorophore. The emitted optical signal can be used to construct an image. The image can be a tomographic image. Furthermore, it is understood that steps (a)-(d) or steps (c)-(d) can be repeated at predetermined intervals thereby to permit evaluation of the subject over time.

The illuminating and/or detecting steps (steps (c) and (d), respectively) can be performed using an endoscope, catheter, tomographic system, planar system, hand-held imaging system, goggles, or an intraoperative imaging system or microscope.

Before or during these steps, a detection system can be positioned around or in the vicinity of a subject (for example, an animal or a human) to detect signals emitted from the subject. The emitted signals can be processed to construct an image, for example, a tomographic image. In addition, the processed signals can be displayed as images either alone or as fused (combined) images.

It will be understood by those of ordinary skill in the art, that in some embodiments, D can represent two or more different fluorophores. For example, D can include a first fluorophore (D1) and second fluorophore (D2) which can be, for example, dyes which are not the same. In other embodiments, D can represent three or four different dye molecules (D1, D2, D3, D4) each linked by a biodegradable linker, which can be the same or different, to a PEP portion of the molecule of the present invention.

In accordance with another embodiment, the present invention provides an imaging agent having the following formula (II):

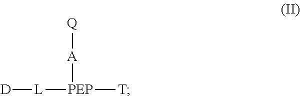

wherein: D is 1 to 4 fluorophores which can be the same or different; L is 1 to 4 enzymatically cleavable peptide linkers which can be the same or different; PEP is a peptide sequence with overall hydrophilicity that both promotes the self-assembly of the designed molecules into nanostructures and facilitates better cell targeting and internalization; A is a side chain moiety of an amino acid of PEP; Q is a fluorescence quencher molecule; and T is a targeting ligand.

Peptide-based targeting ligands including, but not limited to, integrin binding peptides such as RGD, RGDS and similar derivatives, prostate specific membrane antigen (PSMA) ligands, etc, can be directly introduced as part of the peptide sequence (PEP), using the same solid phase Fmoc peptide synthesis techniques.

For example, the following listing of peptides, proteins, and other large molecules may also be used, such as interleukins 1 through 18, including mutants and analogues; interferons α, γ, hormone releasing hormone (LHRH) and analogues, gonadotropin releasing hormone transforming growth factor (TGF); fibroblast growth factor (FGF); tumor necrosis factor-α); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin-a-y-globulin; superoxide dismutase (SOD); and complement factors, and biologically active analogs, fragments, and derivatives of such factors, for example, growth factors.

Members of the transforming growth factor (TGF) supergene family, which are multifunctional regulatory proteins, may be used as the targeting ligand in the DAs of the present invention. Members of the TGF supergene family include the beta transforming growth factors (for example, TGF-131, TGF-132, TGF-133); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (1GF)), (for example, lnhibin A, lnhibin B), growth differentiating factors (for example, GDF-1); and Activins (for example, Activin A, Activin B, Activin AB). Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

Both peptide-based ligands (as described above) and small molecule targeting ligands, including but not limited to, folate-receptor binding molecules such as folate and methotrexate, can be incorporated using common conjugation techniques. These include, but are not limited to, amide bond formation (requiring a lysine, glutamic acid or aspartic acid group at the periphery of the peptide, the C-terminal for instance), reaction with a cysteine thiol (thiol-ene reaction, disulfide formation, thioether formation) or through Click reactions such as azide-alkyne cycloaddition. These conjugations may require suitable modification of the ligand to provide the required functionality, and may be performed on the solid-phase during synthesis of the peptide or in solution once the peptide has been isolated.

In addition, it is possible to practice an in vivo imaging method that selectively detects and images one or more molecular imaging probes, including the imaging agents simultaneously. In such an approach, for example, in step (a) noted above, two or more imaging probes whose signal properties are distinguishable from one another are administered to the subject, either at the same time or sequentially, wherein at least one of the molecular imaging probes is a agent. The use of multiple probes permits the recording of multiple biological processes, functions or targets.

The subject may be a vertebrate, for example, a mammal, for example, a human. The subject may also be a non-vertebrate (for example, C. elegans, drosophila, or another model research organism, etc.) used in laboratory research.

With respect to in vitro imaging, the imaging agents can be used in a variety of in vitro assays. For example, an exemplary in vitro imaging method comprises: (a) contacting a sample, for example, a biological sample, with one or more imaging agents of the invention; (b) allowing the agent(s) to interact with a biological target in the sample; (c) optionally, removing unbound agents; (d) in the case of fluorescent agents, illuminating the sample with light of a wavelength absorbable by a fluorophore of the agents; and (e) detecting a signal emitted from fluorophore thereby to determine whether the agent has been activated by or bound to the biological target.

After an agent has been designed, synthesized, and optionally formulated, it can be tested in vitro by one skilled in the art to assess its biological and performance characteristics. For instance, different types of cells grown in culture can be used to assess the biological and performance characteristics of the agent. Cellular uptake, binding or cellular localization of the agent can be assessed using techniques known in the art, including, for example, fluorescent microscopy, FACS analysis, immunohistochemistry, immunoprecipitation, in situ hybridization and Forster resonance energy transfer (FRET) or fluorescence resonance energy transfer. By way of example, the agents can be contacted with a sample for a period of time and then washed to remove any free agents. The sample can then be viewed using an appropriate detection device such as a fluorescent microscope equipped with appropriate filters matched to the optical properties of a fluorescent agent. Fluorescence microscopy of cells in culture or scintillation counting is also a convenient means for determining whether uptake and binding has occurred. Tissues, tissue sections and other types of samples such as cytospin samples can also be used in a similar manner to assess the biological and performance characteristics of the agents. Other detection methods including, but not limited to flow cytometry, immunoassays, hybridization assays, and microarray analysis can also be used.

As defined herein, in one or more embodiments, “administering” means that the one or more imaging agents of the present invention are introduced into a sample having at least one cell, or population of cells, having a target gene of interest, and appropriate enzymes or reagents, in a test tube, flask, tissue culture, chip, array, plate, microplate, capillary, or the like, and incubated at a temperature and time sufficient to permit uptake of the at least one imaging agents of the present invention into the cytosol.

In another embodiment, the term “administering” means that at least one or more imaging agents of the present invention are introduced into a subject, preferably a subject receiving treatment for a disease, and the at least one or more imaging agents are allowed to come in contact with the one or more disease related cells or population of cells having the target gene of interest in vivo.

These and other aspects of the present invention will be further appreciated upon consideration of the following Examples, which are intended to illustrate certain particular embodiments of the invention but are not intended to limit its scope, as defined by the claims.

### Example 1

### Synthesis of TAT-Based Nanobeacons

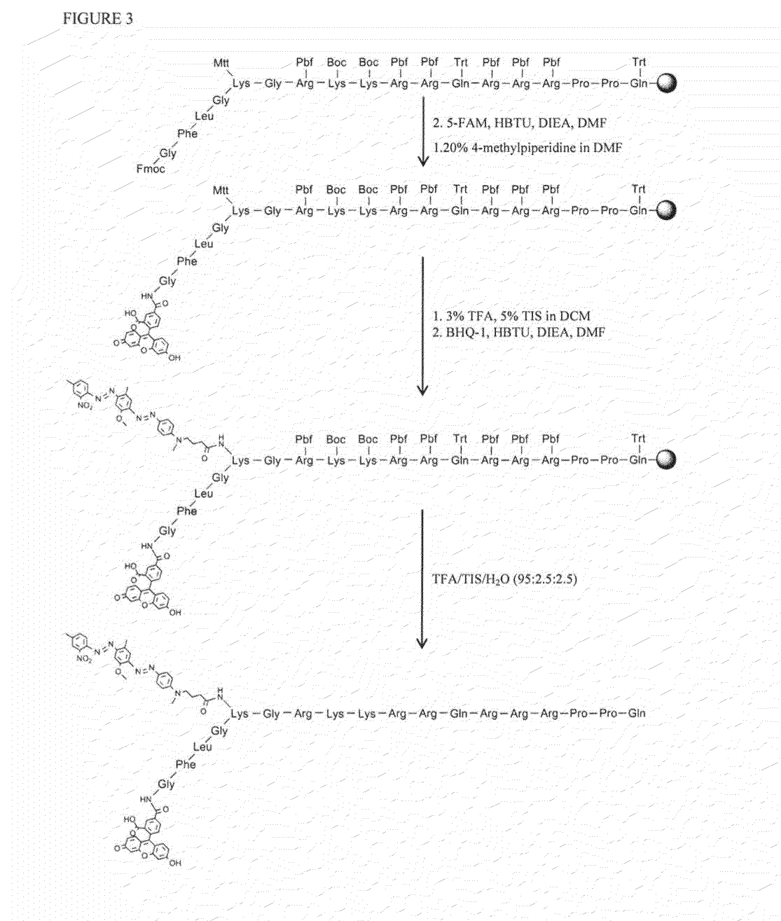

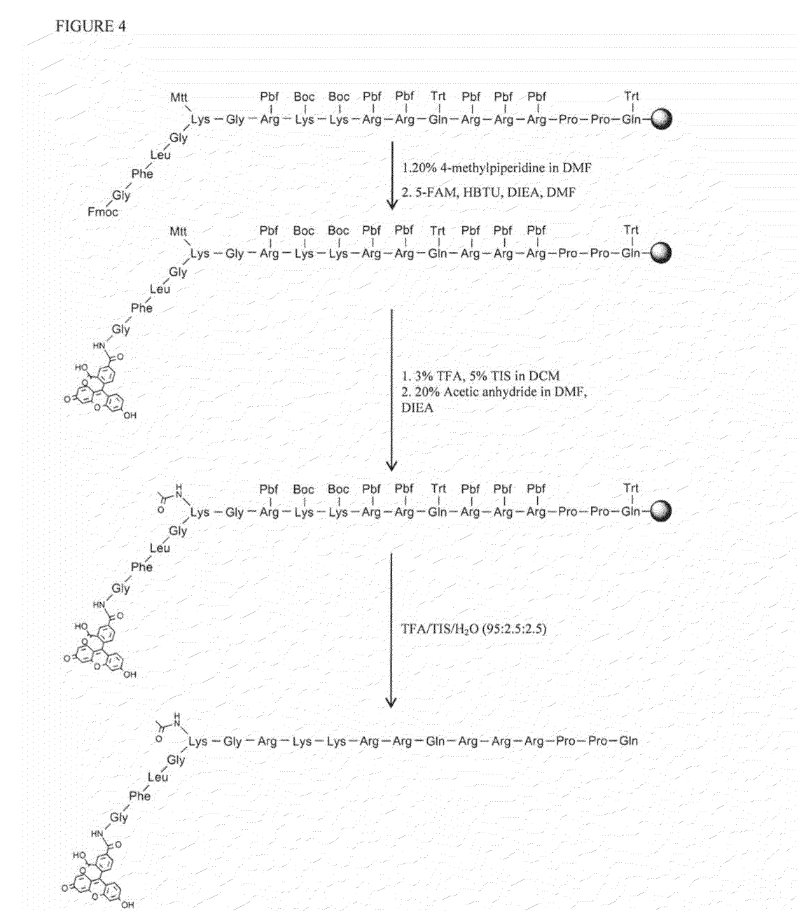

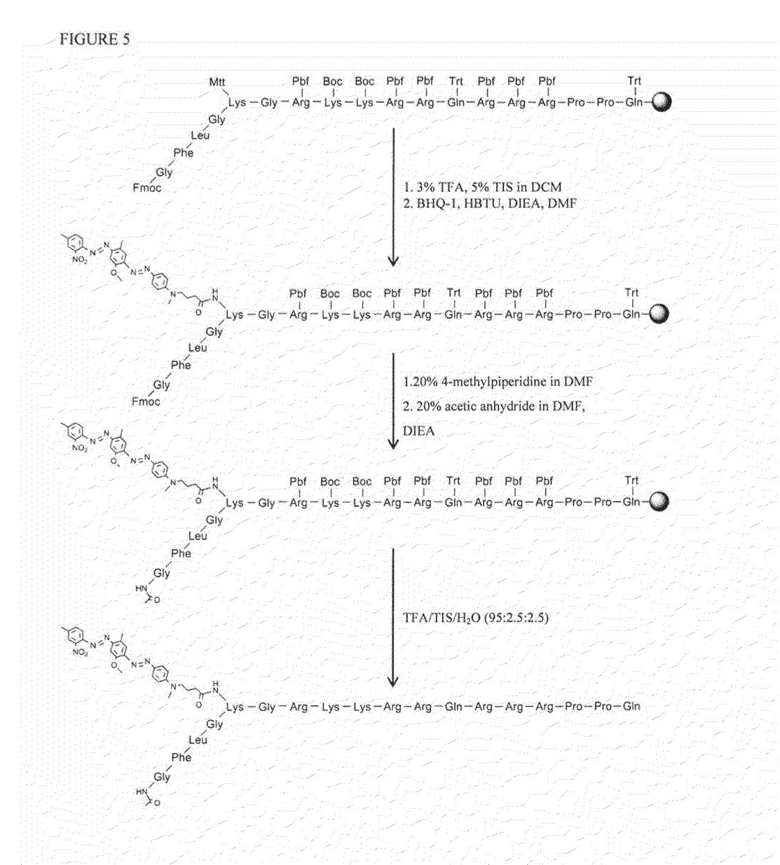

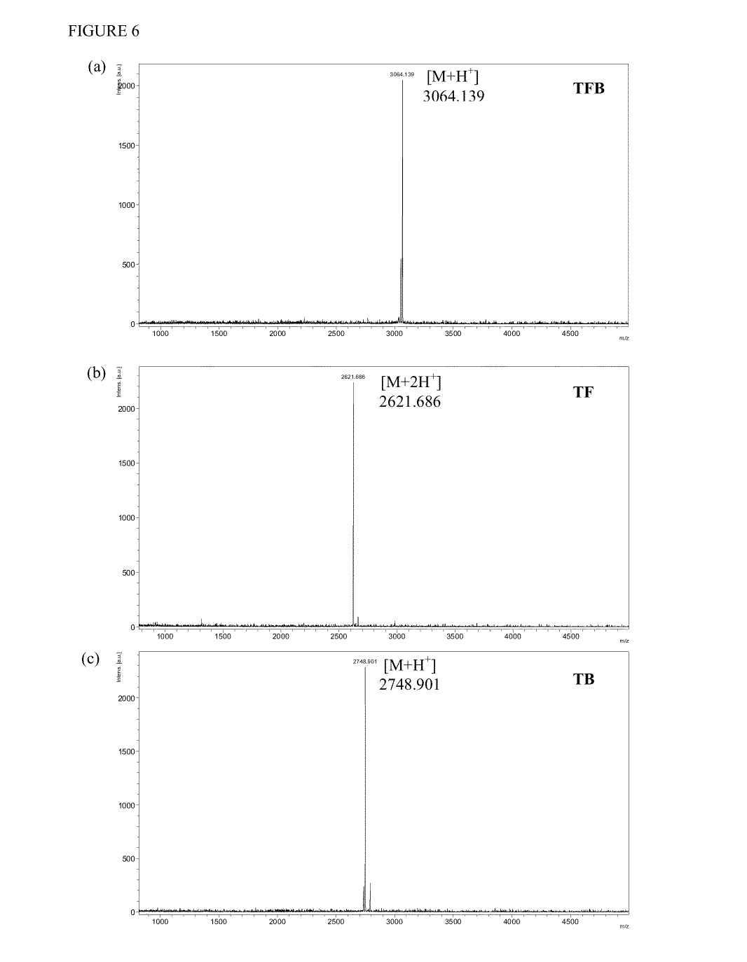

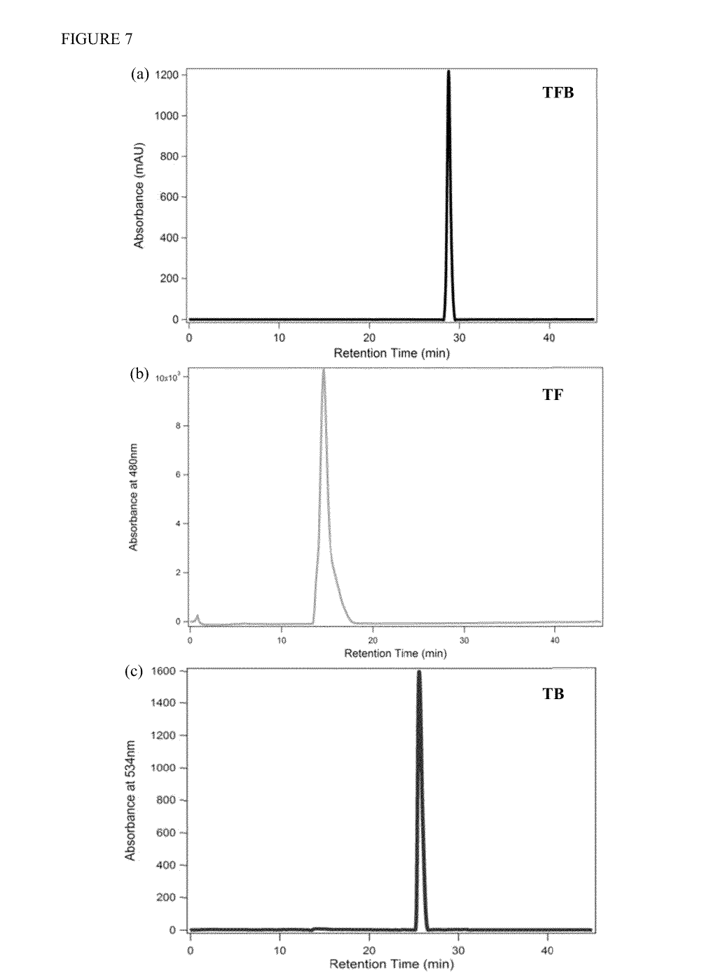

Synthesis. The Tat sequence and the peptide linker (Fmoc-GFLGK(Mtt)GRKKRRQRRRPPQ-Rink) of the TFB molecule was first synthesized on an automatic peptide synthesizer using standard 9-fluorenylmethoxycarbonyl (Fmoc) solid phase synthesis protocols. After removal of the Fmoc protecting group, 5-FAM was manually coupled at the peptide N-terminus. Next, Black Hole Quencher-1 (BHQ-1) was incorporated onto the lysine ε-amine, following removal of the Mtt protecting group for lysine side chains. The completed peptide was cleaved from the Rink Amide resin using a mixture of TFA/TIS/H₂O. The two control molecules: TF and TB were synthesized by using acetic anhydride to replace the BHQ-1 and the 5-FAM segments with an acetyl group, following the same procedures for the synthesis of the TFB molecule (FIGS. 3-5). All the molecules were purified using preparative RP-HPLC and their purity was evaluated by MALDI-TOF mass spectrometry (FIG. 6) and analytical HPLC (FIG. 7)

Purification. The peptides were purified by preparative RP-HPLC using a Varian Polymeric Column (PLRP-S, 100 Å, 10 μm, 150×25 mm) at 25° C. on a Varian ProStar Model 325 preparative HPLC (Agilent Technologies, Santa Clara, Calif.) equipped with a fraction collector. A water/acetonitrile gradient containing 0.1% v/v TFA was used as eluent at a flow rate of 25 mL/min, monitoring the absorbance at 480 nm and 534 nm for TF and TB/TFB molecules respectively. The crude materials were dissolved in 30 ml of 0.1% aqueous TFA, and each purification run was carried out with a 10 ml injection. Collected fractions were analyzed by ESI-MS (LDQ Deca ion-trap mass spectrometer, Thermo Finnigan, San Jose, Calif.) and those containing the desired product were further concentrated by rotary evaporation to remove acetonitrile. The remaining solution was lyophilized (FreeZone −105° C. 4.5 L freeze dryer, Labconco, Kansas City, Mo.) and stored at −30° C.

MALDI-TOF Characterization. High resolution peptide masses were determined by MALDI-TOF mass spectrometry, using a BrukerAutoflex III MALDI-TOF instrument (Billerica, Mass.). Samples were prepared by depositing 1 μL of sinapinic acid matrix (10 mg/ml in 0.05% TFA in H₂O/MeCN (1:1), Sigma-Aldrich, PA) onto the target spot, and allowed to dry for 5 minutes. 1 μL of peptide aqueous solution (0.1% TFA) were deposited on the corresponding spot and quickly mixed with 1 μL of sinapinic acid matrix solution. Samples were irradiated with a 355 nm UV laser and analyzed in the reflectron mode.

Analytical HPLC Characterization. Analytical reverse-phase HPLC was performed using a Varian polymeric column (PLRP-S, 100 Å, 10 nm, 150×4 6 mm) with 20 μL injection volumes. A water/acetonitrile gradient containing 0.1% v/v TFA at a flow rate of 1 mL/min was used and samples were dissolved at 1 mg/ml concentrations in 0.1% aqueous TFA.

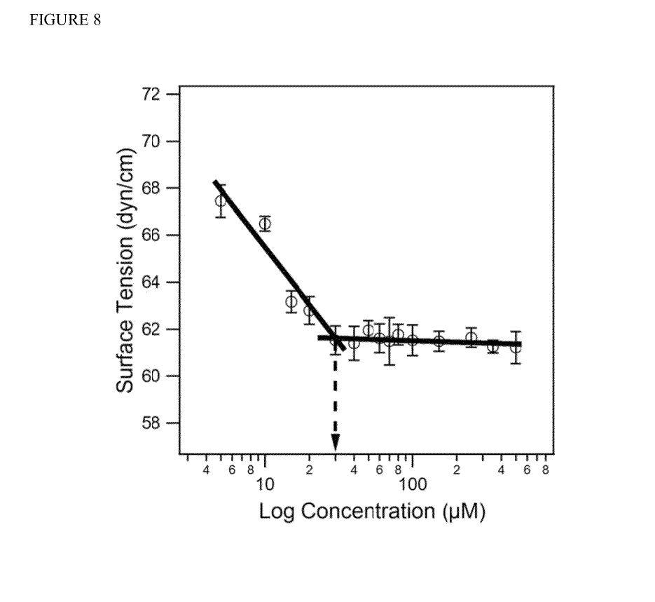

TFB Critical Aggregation Concentration (CAC) Determination. 500 μM TFB stock solutions were prepared in sodium acetate buffer (pH 5) and serial dilution method used to prepare various concentrations of TFB samples with final volumes of 100 μL. All samples were protected from light and left overnight at room temperature. Surface tension measurement was carried out using pendant drop method. Measurement apparatus includes micrometer syringe GE 2.0 mL from Gilmont Instrument, dispensing needle 22 Gauge×0.5″ blunt tip, and series of pendant drop images were taken by First Ten Angstroms (FTA) 125. Surface tension measurements were further analyzed using FTA32 software. As shown in FIG. 8, TFB critical aggregation concentration was determined to be 30 μM at pH 5.

### Example 2

### Verification of Fluorescence Quenching in TFB Nanobeacons

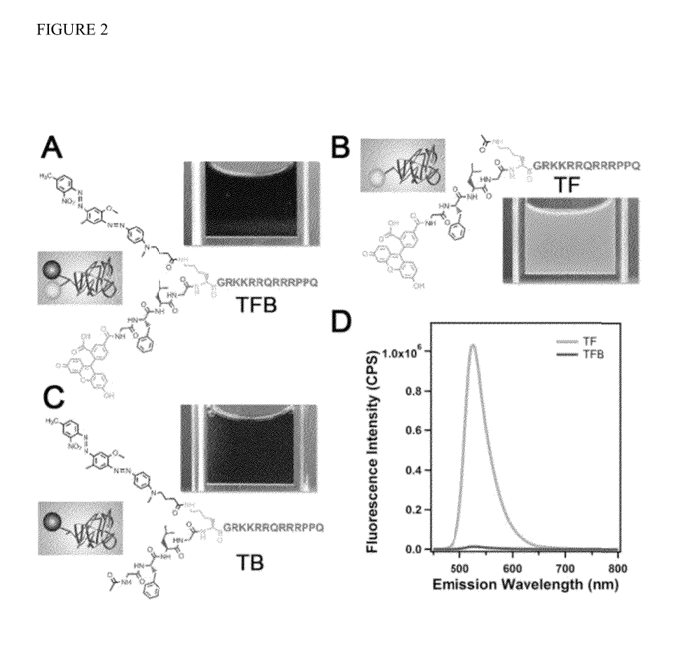

Quenching Effect. We synthesized two control molecules: TF (FIG. 2B) and TB (FIG. 2C) to assist in better understanding of the quenching effect and self-assembly behaviors of the TFB imaging agent molecule. The effective quenching of the 5-FAM fluorophore by BHQ-1 in the imaging agent molecule can be inferred by a change in solution color between three molecules (FIGS. 2A-2C). At a concentration of 200 μM, the aqueous solution of the TF conjugate appears bright green, owing to the 5-FAM fluorescence around 520 nm (FIG. 2B). In contrast, the aqueous solution of 200 μM TB displays a dark red color (FIG. 2C) due to the absorption in the visible light region between 400-650 nm. The dark red color of 200 μM TFB solution (FIG. 2A), similar to that of TB solution but distinct from that of TF solution, strongly suggests an effective quenching of 5-FAM fluorescence. This effective quenching was further supported by the measurements of the fluorescence of the 5-FAM chromophore. It was found that the 5-FAM fluorescence intensity of 1 μM TFB solution drops more than 80 times relative to that of a TF solution of the same molar concentration (FIG. 2D), implying a greater than 98% efficiency of 5-FAM fluorescence resonance energy transfer within the designed imaging agent molecule.

### Example 3

### Self-Assembly Characterization of TFB Nanobeacons

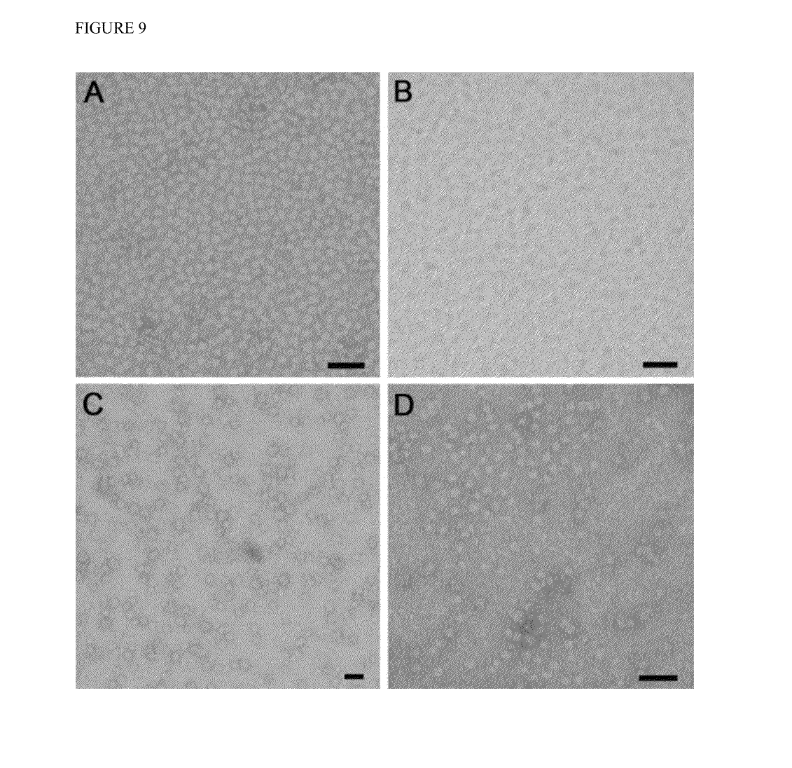

Self-assembly of TFB was initiated by dissolution of the molecule into either Milli-Q water or in phosphate buffered saline (PBS). Transmission electron microscopy (TEM) studies showed that all three molecules, TFB, TF and TB, self-assemble into spherical micelles under physiological conditions, with sizes of 11.1±1.2 nm, 18.4±3.7 nm, and 13.1±1.0 nm, respectively (FIGS. 9A-9D). A representative TEM image from a 1×PBS solution of 200 μM TFB is shown in FIG. 9A, revealing dominant nanoparticles with a uniform size of approximately 11 nm. In this image, the nanoparticles appear brighter than the background due to the use of uranyl acetate as a negative staining agent which deposits more on the background and thus reverses the image contrast. The size and shape of these imaging agent nanoparticles was further confirmed using cryogenic TEM imaging techniques (FIG. 9B) which involve no staining but direct imaging of the liquid sample solution.

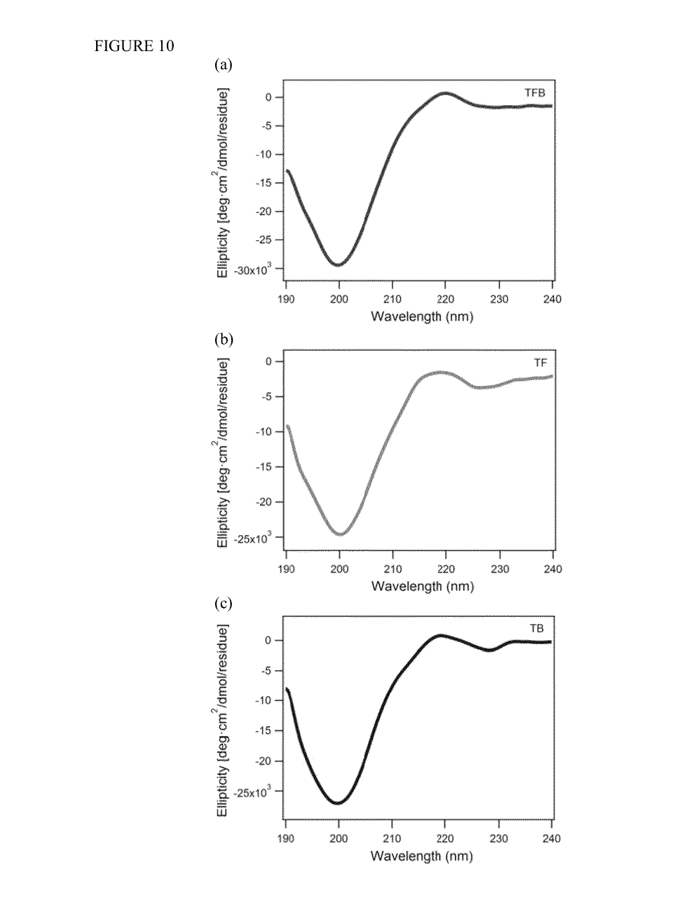

Circular dichroism measurements show that the hydrophilic Tat sequence assumes a random coil secondary structure (FIG. 10). The diameter of 11 nm is reasonably close to twice that of the expected molecular length of TFB. The amphiphilic nature of the TFB leads us to conclude that nanoparticles observed in FIG. 9A are core-shell micelles with the 5-FAM and BHQ-1 segments comprising the core. Since enzyme-catalyzed reactions involve the formation of enzyme substrate complexes, the fact that the -GFLG- (SEQ ID NO: 1) linkers are deeply embedded within the micellar core shows that in the assembled state the -GFLG-(SEQ ID NO: 1) peptide linkers are inaccessible to CatB for cleavage.

### Example 4

### Enzymatic Degradation of TFB Nanobeacons

Enzymatic digestion experiments were carried out to evaluate the degradation kinetics of TFB NBs by CatB. In these experiments, CatB was first activated for 5 minutes at 37° C. using a reaction buffer containing 1 mM EDTA and 25 mM L-cysteine. All solutions were adjusted to pH 5.0 using a 3 M HCl solution to ensure proper CatB function. NB solution was then introduced to solutions containing the desired amount of activated CatB, and the solution fluorescence was subsequently monitored.

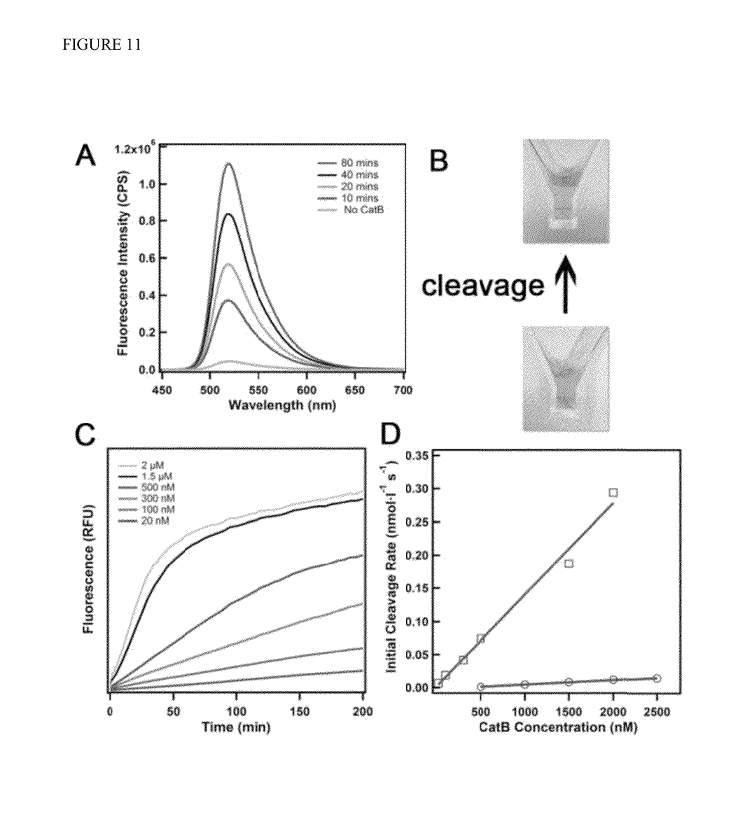

FIG. 11A shows that in the presence of only 1 μM CatB the fluorescence intensity rapidly increases with time, with an approximate 25-fold increase in the peak intensity at 520 nm within 80 minutes. After a sufficient time for cleavage, the solution color was observed to change from light red to light yellow (FIG. 11B). The small fluorescence peak in the absence of CatB arises from incomplete quenching of 5-FAM, and its intensity did not change over time, suggesting that the TFB molecules are rather stable under the experiment conditions. In order to correlate the fluorescence intensity to the enzyme activity and also to understand the enzyme cleavage efficiency on the studied NB molecule, we performed a series of experiments on 1 μM TFB solutions while varying the amount of CatB added. The 1 μM concentration is far below the critical micellization concentration (CMC) of TFB at pH 5, which was determined to be around 30 μM using a surface tension measurement method (FIG. 8). FIG. 11C clearly reveals that an increase in CatB concentration leads to faster cleavage of 5-FAM from TFB. It is also evident that concentrations of CatB as low as 20 nM can effectively cleave the peptide linker, although the reaction proceeds at a much slower rate. We found that the initial rate of cleavage scales linearly with the concentration of CatB (FIG. 11D). The catalytic reaction of CatB has been reported to follow the kinetic behavior described by the Michaelis-Menten equation. According to Michaelis-Menten Equation, the reaction rate V can be expressed in the following form:

$$V = \frac{{{k}_{cat} [ E ]}_{t} [ S ]}{{K}_{M} + [ S ]}$$

in which k_{cat} is the first-order rate constant, [E]ₜ is the total enzyme concentration, [S] is the substrate (TFB, in the case reported here) concentration, and K_{M} is the Michaelis-Menten constant. At the very low substrate concentrations reported herein ([S]<<K_{M}), the equation can be rewritten as:

$$V ≅ {\frac{{k}_{cat}}{{K}_{M}} [ E ]}_{t} [ S ]$$

The ratio of k_{cat}/K_{M} provides a direct measure of enzyme efficiency and specificity. The plot in FIG. 11D is in good agreement with this equation as the initial cleavage rate is indeed linear with respect to the CatB concentration. The initial reaction rates, V₀, were obtained from the linear region at the very beginning of the curves presented in FIG. 11C. k_{cat}/K_{M} was calculated using this simplified Michaelis-Menten equation, and was found to be approximately 137 (mol/L)⁻¹s⁻¹. This value shows a reasonable degradation efficiency of the -GFLG- linker to CatB digestion. This finding also implies that quantitative detection of CatB in live cells is possible once accurate measurements of the initial reaction rate and the local concentration of the delivered NBs can be obtained.

Further experiments were performed on 50 μM TFB solution, a concentration above the CMC (30 μM), to gain a better understanding of the degradation kinetics of TFB micelles. As expected, the cleavage reaction was found to proceed much more slowly, and the k_{cat}/K_{M} was calculated to be around 0.135 (mol/L)⁻¹s⁻¹ (FIG. 11D), a value almost three orders of magnitude lower than that of CatB cleavage on the TFB monomers. Since TFB predominantly exists in aggregates above the CMC, these results clearly show that the -GFLG- peptide linker is inaccessible for effective CatB cleavage, and thus prove the expected cleavage mechanism presented in FIG. 1A.

### Example 5

### In Vitro Cellular Activation of TFB Nanobeacons

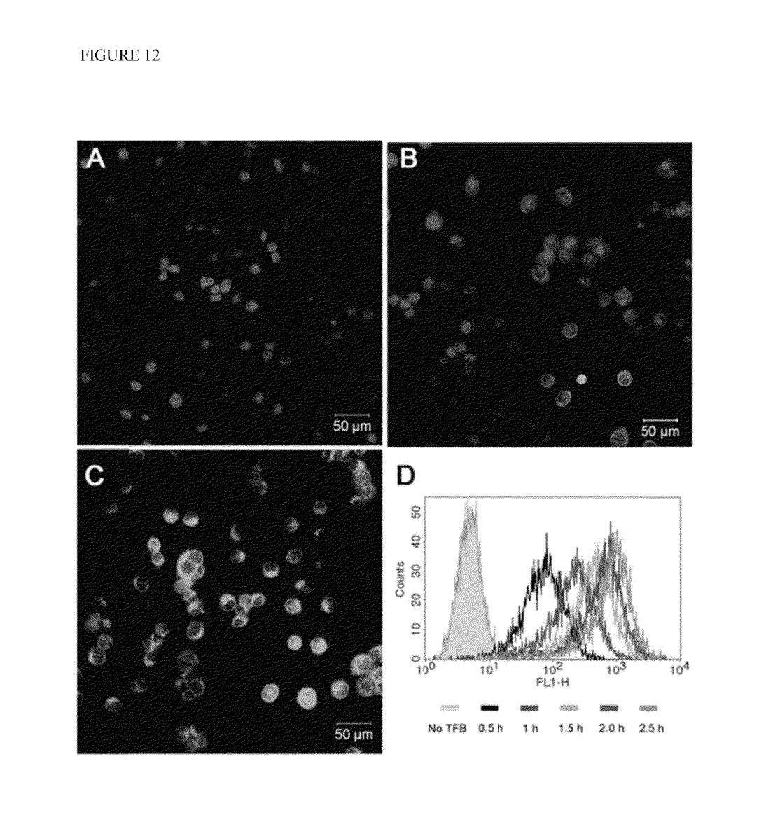

To assess the possibility of using the designed imaging agents for detection of CatB activities in cancerous cells, MCF-7 human breast cancer cells were incubated with 5 μM TFB at 37° C. in cell media, and fluorescence images on the basis of 5-FAM emission were taken at different time points (0 h, 0.5 h, and 1.5 h). The cells were stained with Hoechst 33342 (blue). FIGS. 12 A-C reveal increased 5-FAM fluorescence intensity inside the MCF-7 cells with increased incubation time. Since intact TFB molecules remain dark and are not fluorescent, this result reveals that the imaging agent molecule is not only capable of entering the cells but can also be effectively activated within cells to generate green fluorescence.

To confirm the observed 5-FAM fluorescence does not stem from potential artifacts associated with cell fixation, flow cytometry was used to investigate the time-dependant fluorescence in live cells (FIG. 12D). These results are consistent with the fluorescent imaging data. The continuous increase in fluorescence intensity with prolonged incubation time suggests effective cellular uptake of the imaging agent molecules. It is thought that this effective internalization is a combined effect of using the Tat cell penetrating peptide with the amphiphilic design of the imaging agent molecule. For the TFB concentrations used in these studies, cell viability tests shows the TFB imaging agent has little toxicity to MCF-7 cells during the incubation (data not shown).

### Example 6

### Cellular Localization of TFB Nanobeacons

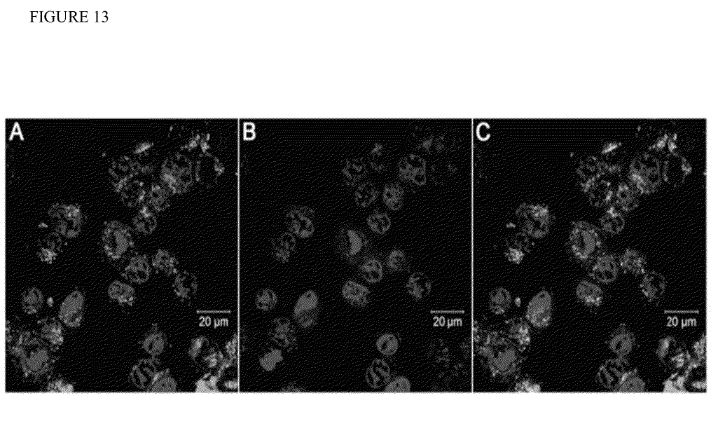

Colocalization experiments were performed to verify the locations from where the 5-FAM fluorescence was generated. Lysosomes of MCF-7 cells were labeled with Lysotracker Red. As shown in FIG. 13, the merged fluorescence image (FIG. 13C) shows almost complete overlap of the 5-FAM green fluorescence with the Lysotracker Red fluorescence, indicating the 5-FAM fluorescence arises from lysosomes where CatB is expected to reside.

### Example 7

### Synthesis of Structure and Surface Charge Tunable Nanobeacons

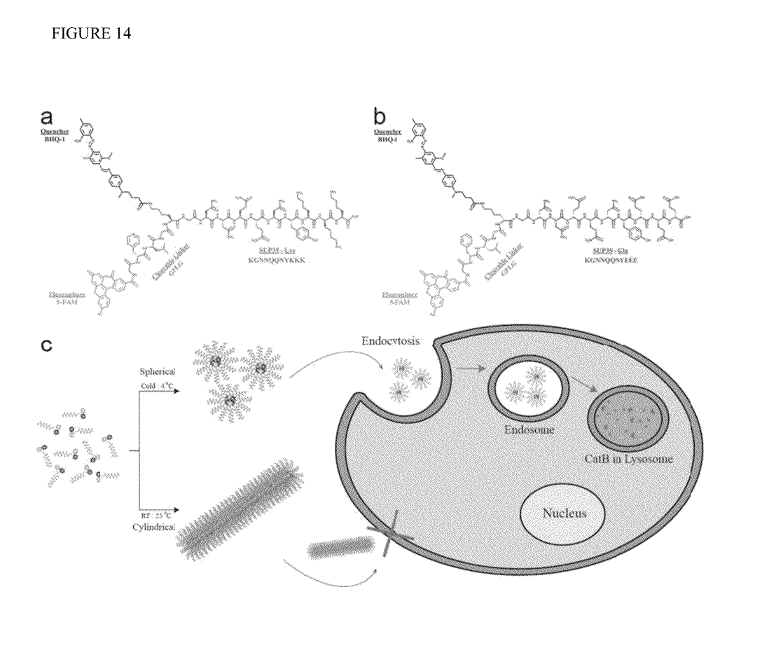

Two peptide amphiphiles of the present invention were designed, namely SFB-K (FIG. 14a) with lysines and SFB-E (FIG. 14b) with glutamic acids serving as the charge source upon ionization of their side chains. An amyloid-forming peptide Sup35 was introduced as the peptide domain sequence to induce one dimensional fiber formation, thereby yielding the cylindrical shape. We also incorporated the beacon concept of fluorophore (5-FAM) and quencher (BHQ-1) pair with an enzyme degradable linker, -GFLG- (SEQ ID NO: 1) tetrapeptide. In the presence of Cathepsin-B, which is a lysosome enzyme overexpressed in numerous cancer cells, cleaves the -GFLG- linker thus releasing 5-FAM fluorophore far away from the BHQ-1 quencher for fluorescence detection.

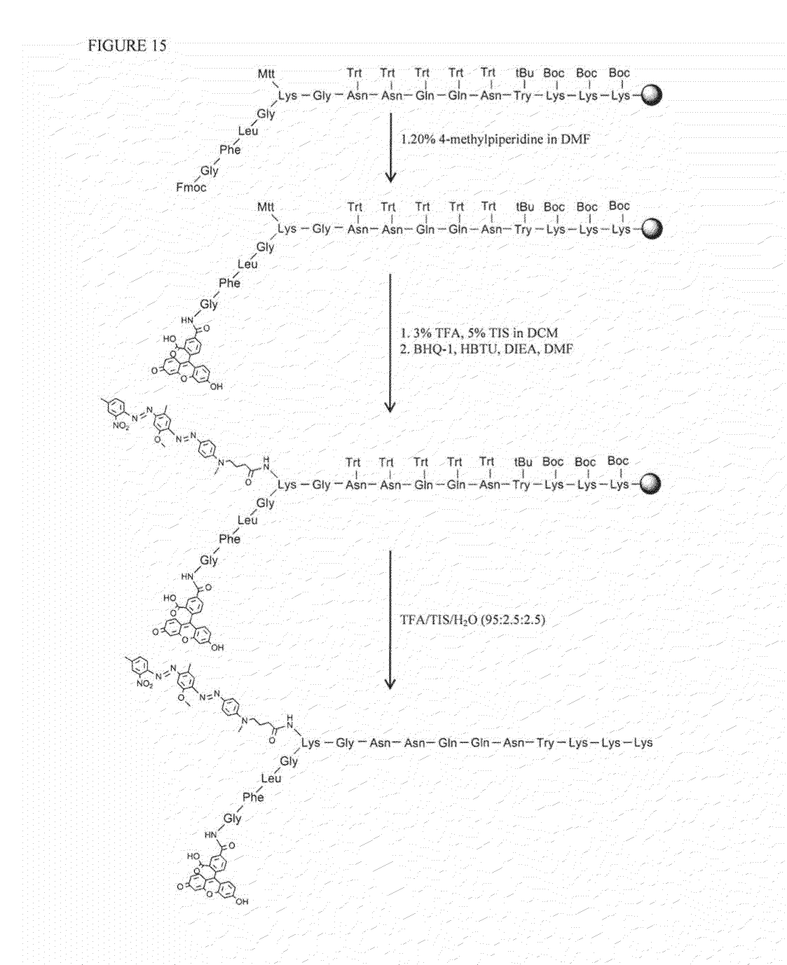

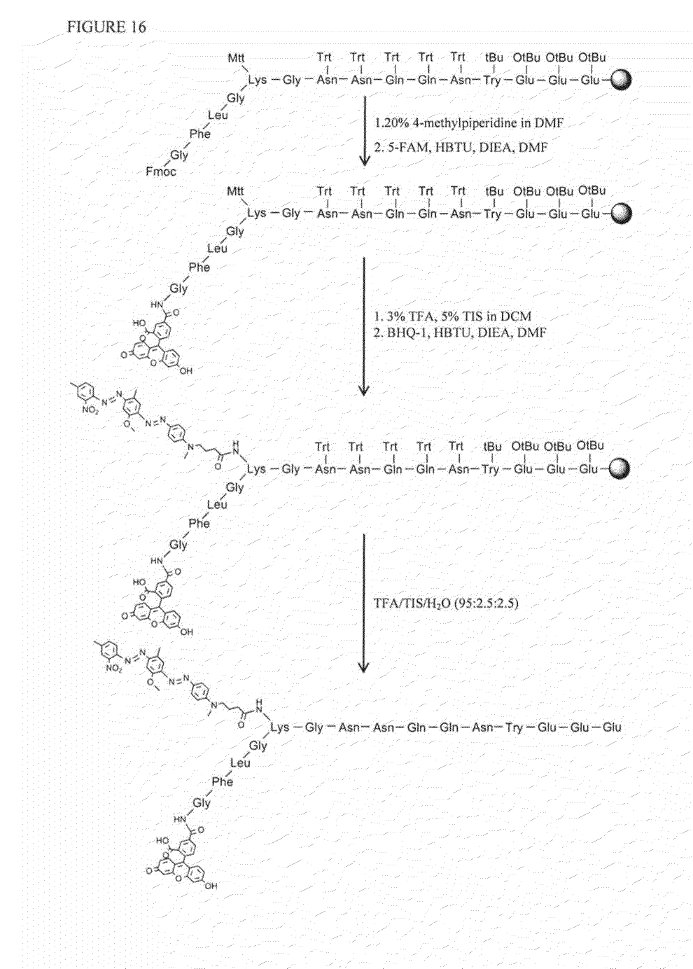

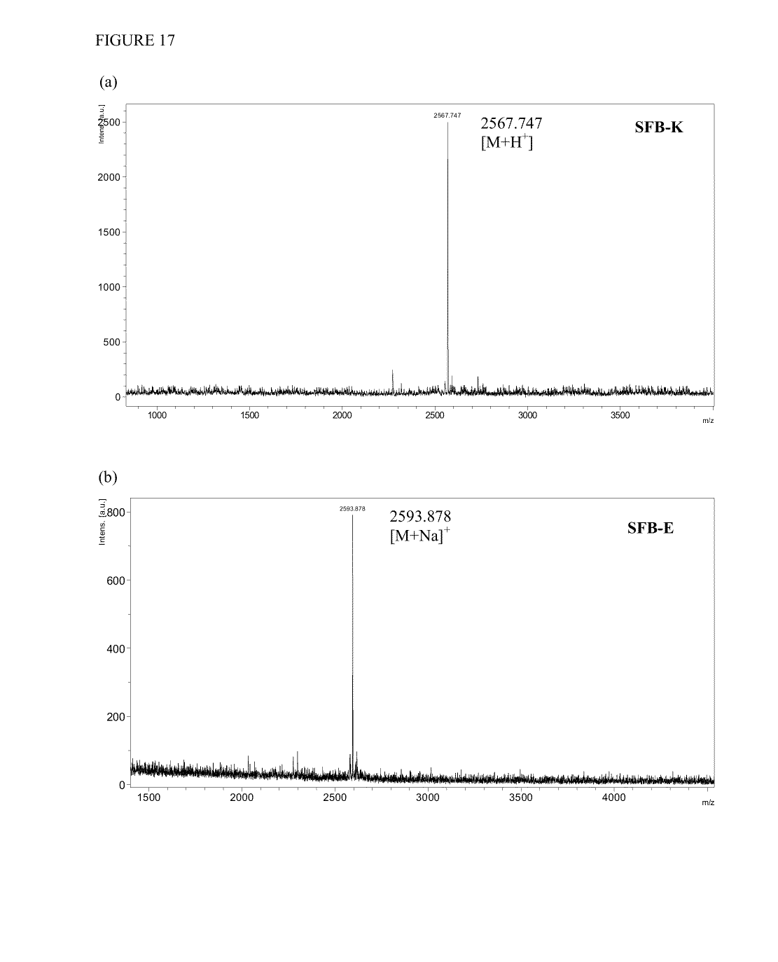

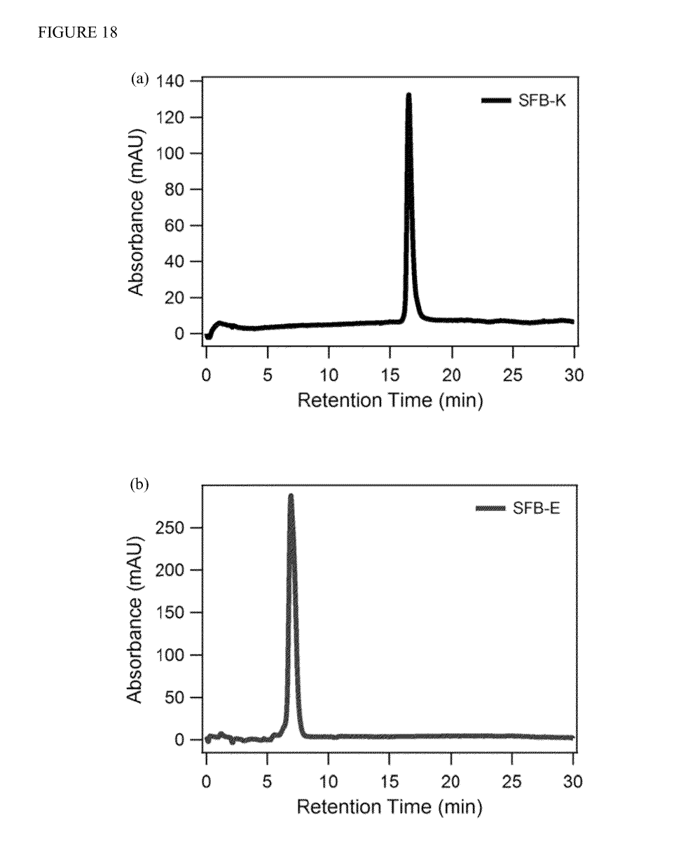

Synthesis of SFB-K and SFB-E nanobeacons. The peptide linker and Sup35 sequence with 3 lysine (Fmoc-GFLGK(Mtt)GNNQQNYKKK-Rink) or 3 glutamic acid (Fmoc-GFLGK(Mtt)GNNQQNYEEE-Wang) of the SFB-K and SFB-E molecules, respectively, were first synthesized on an automatic peptide synthesizer using standard 9-fluorenylmethoxycarbonyl (Fmoc) solid phase synthesis protocols (FIGS. 15-16). After removal of the Fmoc protecting group, 5-FAM was manually coupled at the peptide N-terminus Next, Black Hole Quencher-1 (BHQ-1) was incorporated onto the lysine ε-amine, following removal of the Mtt protecting group for lysine side chains. The completed peptide was cleaved from the Rink Amide resin for SFB-K molecule or Wang resin for SFB-E molecule using a mixture of TFA/TIS/H₂O. All the molecules were purified using preparative RP-HPLC and their purity was evaluated by MALDI-TOF mass spectrometry (FIG. 17) and analytical HPLC (FIG. 18).

### Example 8

### Tunable Self-Assembly of SFB Nanobeacons into Spheres and Cylinders

Nanostructure preparation protocols. Self-assembly of spherical and cylindrical nanostructure/nanobeacons. Hexafluoroisopropanol (HFIP), which is known to break amyloid beta aggregates into a homogenous monomeric form, was used in the following sample preparation procedures to furnish monomeric beacon molecules. SFB-K and SFB-E molecules were first dissolved in HFIP at a concentration of 200 μM and final volume of 200 μL. For SFB-KE, 100 μL of SFB-K (200 μM) and 100 μL SFB-E (200 μM), both in HFIP, were mixed to achieve molecular level mixing at 1:1 equimolar ratio. All SFB-K, SFB-E and SFB-KE samples were prepared in glass vials. Using rotary evaporation, HFIP was removed in 40° C. water bath for 10 minutes, forming a thin film on the wall of the glass vial.

For monomer preparation, all samples were reconstituted in 200 μL of DMSO and kept at room temperature.

For spherical nanostructure formation, all samples were reconstituted in 50 μL 100 mM HEPES buffer and 150 μL of water was added to yield a final sample concentration of 200 μM in 25 mM HEPES buffer. All samples were stored at 4° C.

For cylindrical nanostructure preparation, SFB-K and SFB-KE were dissolved in 50 μL 100 mM HEPES buffer and 150 μL of water was added to yield a final sample concentration of 200 μM in 25 mM of HEPES buffer. These samples were sonicated in a water bath for 20 minutes and stored at room temperature.

SFB-E cylinder formation was accomplished by directly dissolving the lyophilized SFB-E powder in 1×DPBS solution to a final concentration of 200 μM and stored at room temperature. No HFIP pre-treatment was used for this sample.

Transmission Electron Microscopy (TEM) and Cryo-TEM Protocol. Spherical nanostructures (SFB-K, SFB-E and SFB-KE) were aged for 1 day in 4° C. and cylindrical samples were aged for >4 days at room temperature, 5 μL sample was spotted on a carbon film copper grid with 400 square mesh (from EMS: Electron Microscopy Sciences) and wicked away using a filter paper and let it dry it for 10 minutes. 5 μL of 2% uranyl acetate was added to sample grid and wicked away after 10 seconds to form a thin film on the grid. All samples were dried for at least 2 hours before TEM imaging. Cryogenic TEM imaging was also performed on the FEI Tecnai 12 TWIN Transmission Electron Microscope, operating at 80 kV. 3-5 μL of sample solution was placed on a holey carbon film supported on a TEM copper grid (Electron Microscopy Services, Hatfield, Pa.). All the TEM grids used for cryo-TEM imaging were treated with plasma air to render the lacey carbon film hydrophilic. A thin film of the sample solution was produced using the Vitrobot with a controlled humidity chamber (FEI). After loading of the sample solution, the lacey carbon grid was blotted using preset parameters and plunged instantly into a liquid ethane reservoir precooled by liquid nitrogen. The vitrified samples were then transferred to a cryo-holder and cryo-transfer stage which was cooled by liquid nitrogen. To prevent sublimation of vitreous water, the cryo-holder temperature was maintained below −170° C. during the imaging process. All images were recorded by a SIS Megaview III wide-angle CCD camera.

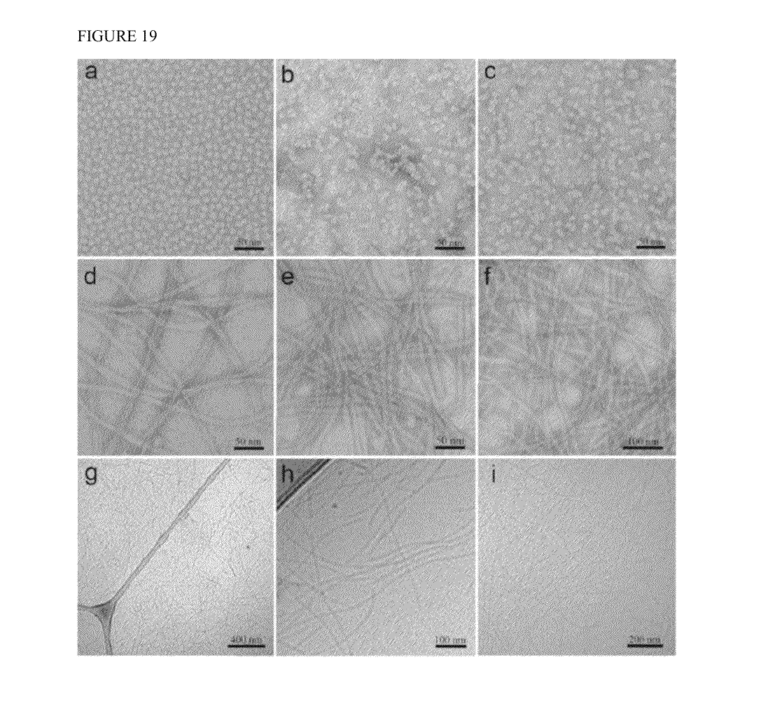

Self-assembly characterization. The self-assembly of SFB nanobeacons into different morphologies were tunable by controlling temperature, solvent and aging days. In order to determine the self-assembly structure formed by SFB-K, SFB-E and SFB-KE, TEM (Transmission Electron Microscopy) and cryo-TEM techniques were used to observe the nanostructure's morphology and diameter. TEM images in FIG. 19(a-c) showed that SFB-K, SFB-E and SFB-KE of 200 μM formed spherical nanostructures after HFIP-rotavap treatment, then reconstituted in 25 mM HEPES buffer. The diameter of spherical SFB-K, SFB-E and SFB-KE are 7.8±0.9 nm, 7.6±1.3 nm and 8.5±1.0 nm, respectively. All samples were kept in 4° C. to maintain its spherical shape. Similarly, SFB-K and SFB-KE were treated with HFIP-rotavap procedure and reconstituted in 25 mM HEPES at room temperature. TEM images in FIG. 19(d & f) showed cylindrical nanostructure of 200 μM SFB-K and SFB-KE after aging for 4 days. SFB-E were dissolved in 1×DPBS directly from purified lyophilized powder form to induce the self-assembly of SFB-E cylindrical nanostructures as shown in FIG. 19(e). From TEM images, the diameter of SFB-K, SFB-E and SFB-KE cylindrical nanostructures are 9.24 nm±1.9 nm, 8.86 nm±1.4 nm and 11.95 nm±1.6 nm, respectively. FIG. 19(g), (h) and (i) showed cryo-TEM of cylindrical SFB-K, SFB-E and SFB-KE in its hydrated form, without any possible distortions from negative staining. The nature of our designed molecule that self-assembled into different shapes with different surface charges, offered an interesting system to study the shape and charge factors affecting cellular uptake.

### Example 9

### Tuning the Surface Charge of SFB Nanobeacons

Surface charge measurements. To determine the charge state of the nanostructure surface, zeta-potential measurements were carried out using a Malvern Zetasizer Nano instrument and its compatible disposable capillary cell (DTS 1070 from Malvern). Spherical and cylindrical SFB nanobeacons were instantly diluted from 200 μM to 5 μM in water, final volume of 1 mL. Measurements were carried in automated mode and repeated three times to obtain the average value and its standard deviation.

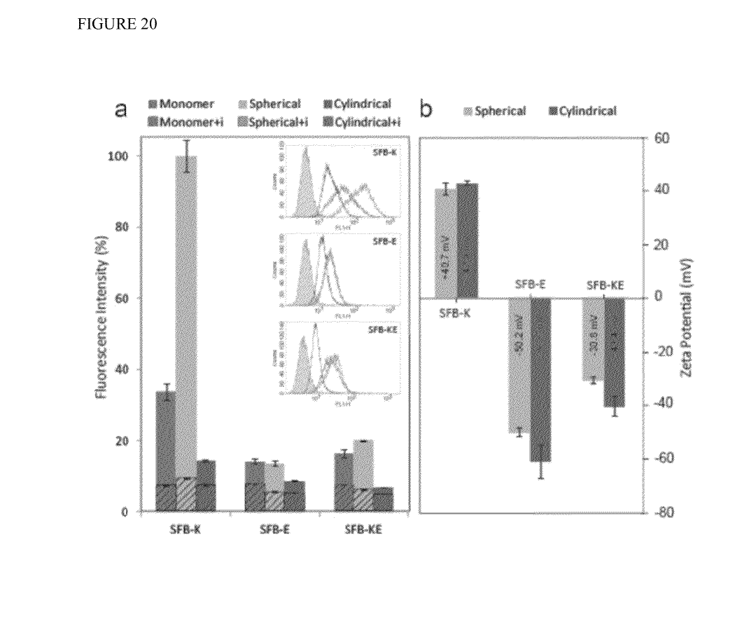

As expected, SFB-K with free amines on the lysine side chain designated positive surface charge of +40.7±2.1 mV and +42.9±0.7 mV for spherical and cylindrical nanostructures, respectively (FIG. 20b). On the other hand, the negative surface charge of SFB-E nanostructures was contributed by the free carboxylic group of glutamic acid's side chain and the C-terminus when they were deprotonated. The zeta potentials for spherical and cylindrical SFB-E were −50.2±1.6 mV and −61.1±6.2 mV, respectively. In a 1:1 mixing ratio of SFB-K and SFB-E, SFB-KE showed negative surface charge of −30.8±1.1 mV and −40.4±3.6 mV for spherical and cylindrical nanostructures. The anionic characteristic of SFB-KE nanostructure was a result of an overall negative charge upon mixing of SFB-E with 4 carboxylic acids and SFB-K with 3 amine groups. These results show that the surface charge of an SFB-based nanobeacon can be tuned through the formation of a catanionic mixture containing the appropriate ratio of SFB-K and SFB-E.

### Example 10

### Enzymatic Activation of SFB Nanobeacons

Enzymatic activation protocol. CatB enzymatic reaction buffer was prepared in 50 mM sodium acetate buffer with 25 mM L-cysteine as enzyme activator and 1 mM EDTA was added as enzyme stabilizer. 0.1 units of CatB was pre-incubated in reaction buffer for 5 minutes, 37° C. to activate the enzyme and SFB nanobeacons were added to reaction buffer to yield a final concentration of 5 μM and final volume of 100 μL. All samples were performed in triplicate and the experiment was carried out in a 96-well standard opaque plate. 5-FAM molecule was excited at 492 nm and emission was collected at 520 nm with 515 nm cut off Using a Gemini XPS microplate reader (Molecular Devices, Sunnyvale, Calif.), the kinetic mode was selected and fluorescence intensity was measured over 125 minutes reading the fluorescence at 5 minute intervals.

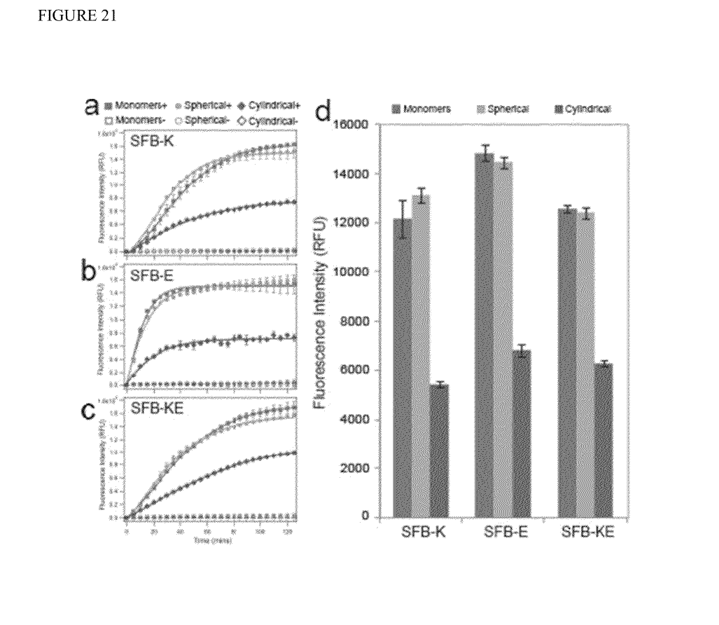

In order to show that the SFB nanobeacons are activatable by Cathepsin-B degradation, 5-FAM fluorescence intensity was monitored after the addition of CatB enzyme. All samples that contained CatB enzyme showed increasing fluorescence intensity over time while samples without CatB remained close to the baseline. This confirms that all synthesized SFB nanobeacons were responsive/activatable by the CatB, as the increase in 5-FAM fluorescence after degradation of the GFLG peptide linker indicates the separation of 5-FAM fluorophore from the BHQ-1 quencher. Comparing the different morphological state of the nanobeacons, it was found that monomeric and spherical SFBs degraded at a similar rate while cylindrical SFBs exhibited slower degradation kinetics. For cylindrical nanobeacons, the intermolecular hydrogen bonding could enhance the stability of nanostructure. Consequently, the dissociation from cylindrical nanostructure to monomers is slower, resulting in a reduced degradation rate. No effect of the surface charge on the degradation rate of monomers and spherical SFB nanobeacons was observed, being similar for SFB-K, SFB-E and SFB-KE as shown by red and green curves in FIGS. 21(a), (b), and (c).

### Example 11

### Effect of Nanostructure Shape and Surface Charge of SFB Nanobeacons on Cellular Uptake

In vitro cellular uptake and inhibition protocols. PC3-Flu cells were seeded onto 24-well plate with cell density of 1×10⁵ cells/well and incubated in 37° C., 5% CO₂ overnight. 5 μM of SFB nanobeacons (monomers, spherical and cylindrical, independently) was prepared by adding 12.5 μL of 200 μM SFB stock solution into 487.5 μL of 1640 cell medium for PC3-Flu. PC3-Flu cells were incubated with the cell medium containing 5 μM of SFB nanobeacons for 1 hour in 37° C. On the other hand, the energy-dependent endocytosis was inhibited by pre-treatment with 10 mM sodium azide and 10 mM 2-deoxy-D-glucose for 15 minutes, followed by 5 μM SFB nanobeacons incubation for 1 hour in 37° C. Cell medium was removed and 200 μL of Gibco 0.25% Trypsin-EDTA (1×), phenol red (Life Technologies Corporation) was added to PC3-Flu cells and incubated for 2 minutes at room temperature. 500 μL of 1640 cell medium was added to each well and cell were re-suspended from the bottom of each well, then transferred into 1.5 mL Eppendoff tube and kept on ice. All cells were centrifuged at 1.7 k RPM for 90 seconds and supernatant was removed. 500 μL cold 1×PBS was added to wash cells and recentrifuged at 1.7 k RPM for 90 seconds. Supernatant was removed and 200 μL of cold 1×PBS was added to resuspend cells, and then transferred into flow-cytometry tube. 10,000 of live cells were gated and fluorescence intensity was detected using flow cytometry (FACSCalibur, BD).

Confocal laser scanning microscopy protocol. PC3-Flu were seeded onto 8-well plate with cell density of 3×10⁴ cells/well and incubated overnight in 37° C. incubator. 7.5 μL of 200 μM SFB nanobeacons were added to 292.5 μL of 1640 cell medium and transferred to each well containing PC3-Flu cells. The cells were kept at 37° C. for 1 hour and medium was removed followed by a quick wash with 300 μL of cell medium without phenyl red. PC3-Flu cells were imaged directly without fixing the cells. The cell nuclei were stained in blue with Hoechst 33342 and lysosome compartments were stained with Lysotracker Red for 30 minutes before the cell imaging.

Cellular internalization of nanobeacon imaging agents. The cellular internalization rates of the self-assembled nanostructures were investigated using PC3-Flu, prostate cancer cell line. All cells were treated with 5 μM of SFB nanobeacon of respective charge and shape for 1 hour. To better illustrate the effect of self-assembled shape in cellular uptake, SFB monomers were included in the in vitro cell study as a control set. The internalization rate of SFB nanobeacon was evaluated by quantifying the released 5-FAM fluorescence through flow cytometry. Our results demonstrated high correlation of cellular internalization towards nanoparticle's surface charge and shape. Spherical SFB-K with positive charge showed highest cellular uptake with ˜3 times faster than its monomeric form and ˜6 times faster than its cylindrical and negative counterparts, as shown in FIG. 20a. The greater cellular uptake rate of positively charged nanoparticles has been reported in several studies and this phenomenon is most likely caused by the electrostatic interaction of cationic nanoparticles with the cell membrane which is slightly anionic. However, the cylindrical nanobeacons did not show any appreciable uptake regardless of its surface charge. We speculate that the elongated cylindrical nanostructures, which possess lengths on the order of micro-meters, are too long and/or large for the cells to take in.

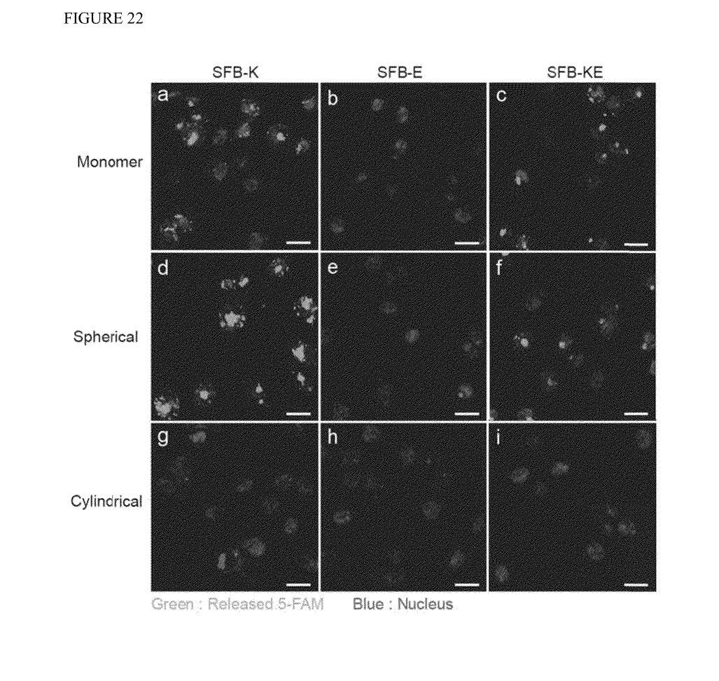

Cellular fluorescence. In order to observe the 5-FAM fluorescence in cell, confocal images of cells were taken after 60 minutes incubation with SFB nanobeacons. PC3-Flu cell nuclei were stained in blue using Hoechst 33342 and the released 5-FAM from SFB nanobeacon would fluoresced in green. Comparable to the flow cytometry results, confocal images showed fastest cellular uptake (brightest green fluorescence) after treated with spherical SFB-K (FIG. 22d) followed by monomeric form of SFB-K (FIG. 22a). Anionic (SFB-E and SFB-KE) and cylindrical shaped nanostructures showed very slow uptake rate as depicted by the low/insignificant 5-FAM green fluorescence (FIG. 22b-c, e-i).

Endocytotic energy. In order to investigate the endocytotic energy dependency of these nanoparticles, PC3-Flu cells were pre-incubated with 10 mM sodium azide (NaN₃) and 10 mM 2-deoxy-D-glucose (DOG) for 15 minutes, followed by 5 μM SFB nanobeacons incubation for 1 hour in 37° C. After the induction of ATP depletion, the cellular uptake was significantly reduced by ˜90% and ˜78% for spherical and monomeric form of SFB-K. A decreased in cellular uptake was observed for all types of molecules (FIG. 20a), indicating the importance of energy dependent in this internalization process.

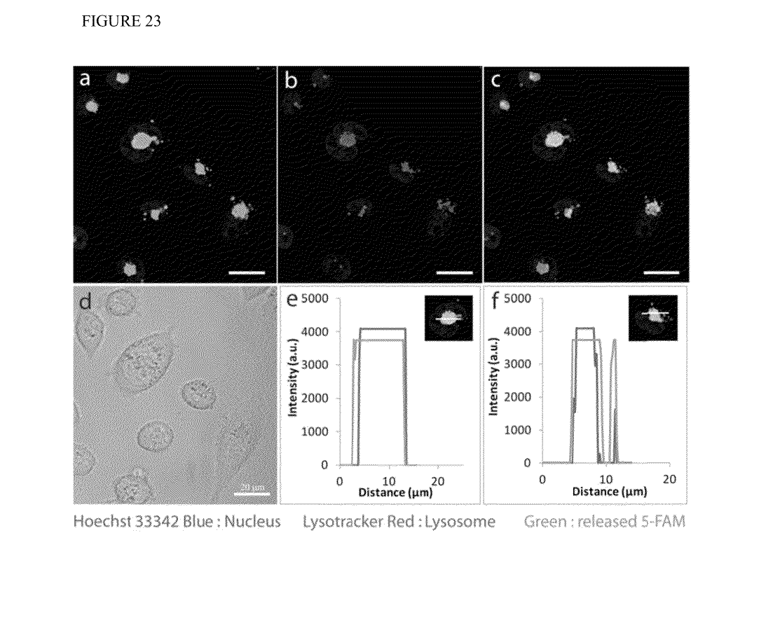

To better understand the internalization of the spherical SFB-K nanoparticles, PC3-Flu cells were pre-treated with Lysotracker Red to label the lysosomal compartments in cell. The merged image of 5-FAM green fluorescence (FIG. 23a) and lysotracker red (FIG. 23b) showed in yellow/orange (FIG. 23c) indicated the co-localization of 5-FAM in the lysosome. The fluorescence intensity of 5-FAM and lysotracker red across PC3-Flu cell was quantified in FIG. 23e-f and the overlap coefficient, R was determined to be 0.9, which indicated high correlation of 5-FAM in the lysosomal compartment.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An imaging agent having the following formula: 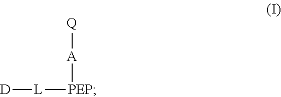
wherein:
D is 1 to 4 fluorophores which can be the same or different;
L is 1 to 4 enzymatically cleavable peptide linkers which can be the same or different;
PEP is a peptide sequence with overall hydrophilicity that both promotes the self-assembly of the designed molecules into nanostructures and facilitates cell targeting and internalization;
A is a side chain moiety of an amino acid of PEP; and
Q is a fluorescence quencher molecule.

2. The imaging agent of claim 1, wherein D is chemically linked either directly or indirectly to a separate amino acid of the enzymatically cleavable oligopeptide.

3. The imaging agent of claim 1, wherein L is an enzymatically cleavable peptide having between 4 and 20 amino acids.

4. The imaging agent of claim 1, wherein L is an enzymatically cleavable peptide having the amino acid sequence GFLG (SEQ ID NO: 1).

5. The imaging agent of claim 1, wherein PEP is a fragment of the HIV Tat protein.

6. The imaging agent of claim 1, wherein A is a lysine chemically linked directly to PEP.

7. The imaging agent of claim 1, wherein Q is chemically linked directly to at least one separate side chain moiety of an amino acid of PEP.

8. The imaging agent of claim 2, wherein D comprises a fluorescent dye.

9. The imaging agent of claim 7, wherein Q is a non-fluorescent quencher.

10. An imaging agent having the following formula: 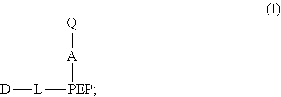
wherein:
D is 5-carboxyfluorescein (5-FAM);
L is an enzymatically cleavable peptide linker having the amino acid sequence GFLG (SEQ ID NO: 1);
PEP is a hydrophilic cell penetrating peptide sequence comprising amino acids 48-60 of the HIV Tat protein;
A is a lysine chemically linked directly to PEP; and
Q is Black Hole Quencher 1 (BHQ1).

11. A method of identifying a cell or a population of cells in vivo expressing a protease of interest comprising:
a) contacting the cell or a population of cells expressing a protease of interest with the imaging agent of claim 1, which is selectively cleavable by a protease of interest;
b) allowing the imaging agent to be selectively cleaved the protease of interest in the cell or population of cells; and
c) detecting the presence of the fluorescent imaging agent after being cleaved by the protease of interest in the cell or population of cells.

12. The method of claim 11, wherein the cell or population of cells is a tumor cell.

13. A method of diagnosing a disease in a patient comprising:
a) administering to a patient suspected of having said disease, an imaging agent which is selectively cleavable by a protease of interest, the cleavage of which indicates the presence of the disease, wherein said imaging agent is an imaging agent of claim 1;
b) allowing the imaging agent to be cleaved by the protease of interest;
c) detecting the presence of the imaging agent binding the protease of interest in the patient.

14. The method of claim 13, wherein the protease of interest is associated with tumor growth and the disease is cancer.

15. A method of identifying a cell or a population of cells in vivo expressing a cathepsin B comprising:
a) contacting the cell or a population of cells expressing cathepsin B with imaging agent having the following formula: 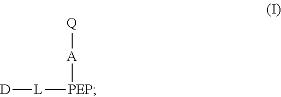
wherein:
D is 5-carboxyfluorescein (5-FAM);
L is an enzymatically cleavable peptide linker having the sequence GFLG;
PEP is a hydrophilic cell penetrating peptide sequence comprising amino acids 48-60 of the HIV Tat protein;
A is a lysine chemically linked directly to PEP; and
Q is Black Hole Quencher 1 (BHQ1);
b) allowing the imaging agent to be selectively cleaved by cathepsin B in the cell or population of cells; and
c) detecting the presence of the fluorescent imaging agent after being cleaved by cathepsin B in the cell or population of cells.

16. An imaging agent having the following formula (II): 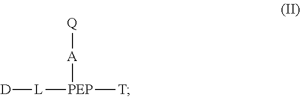
wherein: D is 1 to 4 fluorophores which can be the same or different; L is 1 to 4 enzymatically cleavable peptide linkers which can be the same or different; PEP is a peptide sequence with overall hydrophilicity that both promotes the self-assembly of the designed molecules into nanostructures and facilitates better cell targeting and internalization; A is a side chain moiety of an amino acid of PEP; Q is a fluorescence quencher molecule; and T is a targeting ligand.

17. A method of identifying a cell or a population of cells in vivo expressing a protease of interest comprising:
a) contacting the cell or a population of cells expressing a protease of interest with the imaging agent of claim 16, which is selectively cleavable by a protease of interest;
b) allowing the imaging agent to be selectively cleaved the protease of interest in the cell or population of cells; and
c) detecting the presence of the fluorescent imaging agent after being cleaved by the protease of interest in the cell or population of cells.

18. The method of claim 17, wherein the cell or population of cells is a tumor cell.

19. A method of diagnosing a disease in a patient comprising:
a) administering to a patient suspected of having said disease, an imaging agent which is selectively cleavable by a protease of interest, the cleavage of which indicates the presence of the disease, wherein said imaging agent is an imaging agent of claim 16;
b) allowing the imaging agent to be cleaved by the protease of interest;
c) detecting the presence of the imaging agent binding the protease of interest in the patient.

20. The method of claim 19, wherein the protease of interest is associated with tumor growth and the disease is cancer.
